SEQUENCE LISTING (76 sequences)

SEQ ID NO: 1 - protein, 735 aa, synthetic construct
  REGION 1..735: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQRSTRLALGETTRPAAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 2 - protein, 743 aa, synthetic construct
  REGION 1..743: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNLALGETTRPAQNLQRGNRQAATAAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - protein, 735 aa, synthetic construct
  REGION 1..735: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNLQRGLALGETTRPANRQAATATYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 4 - protein, 10 aa, synthetic construct
  REGION 1..10: note = Made in Lab - amino acid substitution fragment
LALGETTRPA

SEQ ID NO: 5 - protein, 6 aa, synthetic construct
  REGION 1..6: note = Made in Lab - amino acid substitution fragment
RGNRQA

SEQ ID NO: 6 - protein, 8 aa, synthetic construct
  REGION 1..8: note = Made in Lab - amino acid substitution fragment
RGNRQAAP

SEQ ID NO: 7 - protein, 12 aa, synthetic construct
  REGION 1..12: note = Made in Lab - amino acid substitution fragment
NLQRGNRQAATA

SEQ ID NO: 8 - protein, 14 aa, synthetic construct
  REGION 1..14: note = Made in Lab - amino acid substitution fragment
NLQRGNRQAATAAP

SEQ ID NO: 9 - protein, 15 aa, synthetic construct
  REGION 1..15: note = Made in Lab - amino acid substitution fragment
TNQNLQRGNRQAATA

SEQ ID NO: 10 - protein, 17 aa, synthetic construct
  REGION 1..17: note = Made in Lab - amino acid substitution fragment
TNQNLQRGNRQAATAAP

SEQ ID NO: 11 - protein, 7 aa, synthetic construct
  REGION 1..7: note = Made in Lab - amino acid substitution fragment
LGETTRP

SEQ ID NO: 12 - protein, 7 aa, synthetic construct
  REGION 1..7: note = Made in Lab - amino acid substitution fragment
QRGNRQA

SEQ ID NO: 13 - protein, 22 aa, synthetic construct
  REGION 1..22: note = Made in Lab - amino acid substitution fragment
NLQRGNRQAATALALGETTRPA

SEQ ID NO: 14 - protein, 22 aa, synthetic construct
  REGION 1..22: note = Made in Lab - amino acid substitution fragment
NLQRGNRQAATLALGETTRPAA

SEQ ID NO: 15 - protein, 22 aa, synthetic construct
  REGION 1..22: note = Made in Lab - amino acid substitution fragment
NLQRGNRQAALALGETTRPATA

SEQ ID NO: 16 - protein, 22 aa, synthetic construct
  REGION 1..22: note = Made in Lab - amino acid substitution fragment
NLQRGNRQALALGETTRPAATA

SEQ ID NO: 17 - protein, 22 aa, synthetic construct
  REGION 1..22: note = Made in Lab - amino acid substitution fragment
NLQRGNRQLALGETTRPAAATA

SEQ ID NO: 18 - protein, 22 aa, synthetic construct
  REGION 1..22: note = Made in Lab - amino acid substitution fragment
NLQRGNRLALGETTRPAQAATA

SEQ ID NO: 19 - protein, 22 aa, synthetic construct
  REGION 1..22: note = Made in Lab - amino acid substitution fragment
NLQRGNLALGETTRPARQAATA

SEQ ID NO: 20 - protein, 22 aa, synthetic construct
  REGION 1..22: note = Made in Lab - amino acid substitution fragment
NLQRGLALGETTRPANRQAATA

SEQ ID NO: 21 - protein, 22 aa, synthetic construct
  REGION 1..22: note = Made in Lab - amino acid substitution fragment
NLQRLALGETTRPAGNRQAATA

SEQ ID NO: 22 - protein, 22 aa, synthetic construct
  REGION 1..22: note = Made in Lab - amino acid substitution fragment
NLQLALGETTRPARGNRQAATA

SEQ ID NO: 23 - protein, 22 aa, synthetic construct
  REGION 1..22: note = Made in Lab - amino acid substitution fragment
NLLALGETTRPAQRGNRQAATA

SEQ ID NO: 24 - protein, 22 aa, synthetic construct
  REGION 1..22: note = Made in Lab - amino acid substitution fragment
NLALGETTRPALQRGNRQAATA

SEQ ID NO: 25 - protein, 22 aa, synthetic construct
  REGION 1..22: note = Made in Lab - amino acid substitution fragment
LALGETTRPANLQRGNRQAATA

SEQ ID NO: 26 - protein, 6 aa, synthetic construct
  REGION 1..6: note = Made in Lab - Altered AAV capsid protein sequence
SSTTAP

SEQ ID NO: 27 - protein, 4 aa, synthetic construct
  REGION 1..4: note = Made in Lab - Altered AAV capsid protein sequence
SSTT

SEQ ID NO: 28 - protein, 745 aa, synthetic construct
  REGION 1..745: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - protein, 725 aa, synthetic construct
  REGION 1..725: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTRAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 30 - protein, 725 aa, synthetic construct
  REGION 1..725: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQRSTRAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 31 - protein, 725 aa, synthetic construct
  REGION 1..725: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQRGNRQATTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 32 - protein, 725 aa, synthetic construct
  REGION 1..725: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNLQRGNRQAATATYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 33 - protein, 735 aa, synthetic construct
  REGION 1..735: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQRGLALGETTRPANRQATTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 34 - protein, 735 aa, synthetic construct
  REGION 1..735: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNLQRGNLALGETTRPARQAATATYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 35 - protein, 737 aa, synthetic construct
  REGION 1..737: note = Made in Lab - Altered AAV capsid protein sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNLALGETTRPAQRGNRQAAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 36 - protein, 118 aa, synthetic construct
  REGION 1..118: note = Made in Lab - synthetic antibody construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTL

SEQ ID NO: 37 - protein, 110 aa, synthetic construct
  REGION 1..110: note = Made in Lab - synthetic antibody construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 38 - protein, 118 aa, synthetic construct
  REGION 1..118: note = Made in Lab - synthetic antibody construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTL

SEQ ID NO: 39 - protein, 110 aa, synthetic construct
  REGION 1..110: note = Made in Lab - synthetic antibody construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTV

SEQ ID NO: 40 - protein, 458 aa, synthetic construct
  REGION 1..458: note = Made in Lab - synthetic antibody construct
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 725 aa, Adeno-associated virus-5
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPTTGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 42 - protein, 450 aa, synthetic construct
  REGION 1..450: note = Made in Lab
MVSYWDTGVLLCALLSCLLLTGSSSGSKLKDPELSLKGTQHIAGQTLHLQCRGEAAMQHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQ

SEQ ID NO: 43 - protein, 106 aa, synthetic construct
  REGION 1..106: note = Made in Lab
IYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTI

SEQ ID NO: 44 - protein, 15 aa, synthetic construct
  REGION 1..15: note = Made in Lab - Altered AAV capsid protein sequence
STNLQRGNRQAATAD

SEQ ID NO: 45 - protein, 15 aa, synthetic construct
  REGION 1..15: note = Made in Lab - Altered AAV capsid protein sequence
ATNNQSSRRAPATGT

SEQ ID NO: 46 - protein, 15 aa, synthetic construct
  REGION 1..15: note = Made in Lab - Altered AAV capsid protein sequence
ATNNQSSTTAPTTGT

SEQ ID NO: 47 - protein, 15 aa, synthetic construct
  REGION 1..15: note = Made in Lab - Altered AAV capsid protein sequence
ATNNQRSTTAPTTGT

SEQ ID NO: 48 - protein, 15 aa, synthetic construct
  REGION 1..15: note = Made in Lab - Altered AAV capsid protein sequence
ATNNQSSTRAPTTGT

SEQ ID NO: 49 - protein, 15 aa, synthetic construct
  REGION 1..15: note = Made in Lab - Altered AAV capsid protein sequence
ATNNQRSTRAPTTGT

SEQ ID NO: 50 - protein, 15 aa, synthetic construct
  REGION 1..15: note = Made in Lab - Altered AAV capsid protein sequence
ATNNQRGNRQATTGT

SEQ ID NO: 51 - protein, 17 aa, synthetic construct
  REGION 1..17: note = Made in Lab - Altered AAV capsid protein sequence
ATNNQRGNRQAAPTTGT

SEQ ID NO: 52 - protein, 15 aa, synthetic construct
  REGION 1..15: note = Made in Lab - Altered AAV capsid protein sequence
ATNLQRGNRQAATAT

SEQ ID NO: 53 - protein, 23 aa, synthetic construct
  REGION 1..23: note = Made in Lab - Altered AAV capsid protein sequence
ATNNQNLQRGNRQAATAAPTTGT

SEQ ID NO: 54 - protein, 25 aa, synthetic construct
  REGION 1..25: note = Made in Lab - Altered AAV capsid protein sequence
STNLQRGNLALGETTRPARQAATAD

SEQ ID NO: 55 - protein, 25 aa, synthetic construct
  REGION 1..25: note = Made in Lab - Altered AAV capsid protein sequence
ATNNQSSTLALGETTRPATAPTTGT

SEQ ID NO: 56 - protein, 25 aa, synthetic construct
  REGION 1..25: note = Made in Lab - Altered AAV capsid protein sequence
ATNNQSSTTLALGETTRPAAPTTGT

SEQ ID NO: 57 - protein, 25 aa, synthetic construct
  REGION 1..25: note = Made in Lab - Altered AAV capsid protein sequence
ATNNQSSLALGETTRPATTAPTTGT

SEQ ID NO: 58 - protein, 25 aa, synthetic construct
  REGION 1..25: note = Made in Lab - Altered AAV capsid protein sequence
ATNNQSLALGETTRPASTTAPTTGT

SEQ ID NO: 59 - protein, 25 aa, synthetic construct
  REGION 1..25: note = Made in Lab - Altered AAV capsid protein sequence
ATNNQLALGETTRPASSTTAPTTGT

SEQ ID NO: 60 - protein, 25 aa, synthetic construct
  REGION 1..25: note = Made in Lab - Altered AAV capsid protein sequence
ATNNLALGETTRPAQSSTTAPTTGT

SEQ ID NO: 61 - protein, 25 aa, synthetic construct
  REGION 1..25: note = Made in Lab - Altered AAV capsid protein sequence
ATNNQRSTRLALGETTRPAAPTTGT

SEQ ID NO: 62 - protein, 25 aa, synthetic construct
  REGION 1..25: note = Made in Lab - Altered AAV capsid protein sequence
ATNNLALGETTRPAQRSTRAPTTGT

SEQ ID NO: 63 - protein, 25 aa, synthetic construct
  REGION 1..25: note = Made in Lab - Altered AAV capsid protein sequence
ATNNQRGNLALGETTRPARQATTGT

SEQ ID NO: 64 - protein, 27 aa, synthetic construct
  REGION 1..27: note = Made in Lab - Altered AAV capsid protein sequence
ATNNQRGNLALGETTRPARQAAPTTGT

SEQ ID NO: 65 - protein, 25 aa, synthetic construct
  REGION 1..25: note = Made in Lab - Altered AAV capsid protein sequence
ATNLQRGNLALGETTRPARQAATAT

SEQ ID NO: 66 - protein, 25 aa, synthetic construct
  REGION 1..25: note = Made in Lab - Altered AAV capsid protein sequence
ATNLQRGLALGETTRPANRQAATAT

SEQ ID NO: 67 - protein, 27 aa, synthetic construct
  REGION 1..27: note = Made in Lab - Altered AAV capsid protein sequence
ATNNLALGETTRPAQRGNRQAAPTTGT

SEQ ID NO: 68 - protein, 33 aa, synthetic construct
  REGION 1..33: note = Made in Lab - Altered AAV capsid protein sequence
ATNNLALGETTRPAQNLQRGNRQAATAAPTTGT

SEQ ID NO: 69 - protein, 42 aa, Adeno-associated virus 2
PVATEQYGSVSTNLQRGNRQAATADVNTQGVLPGMVWQDRDV

SEQ ID NO: 70 - protein, 42 aa, Adeno-associated virus 1
PVATERFGTVAVNFQSSSTDPATGDVHAMGALPGMVWQDRDV

SEQ ID NO: 71 - protein, 42 aa, Adeno-associated virus 5
RVAYNVGGQWMATNNQSSTTAPATGTYNLQEIVPGSVMERDV

SEQ ID NO: 72 - protein, 42 aa, Adeno-associated virus 6
PVATERFGTVAVNLQSSSTDPATGDVHVMGALPGMVWQDRDV

SEQ ID NO: 73 - protein, 42 aa, Adeno-associated virus 7
PVATEEYGIVSSNLQAANTAAQTQVVNNQGALPGMVWQNRDV

SEQ ID NO: 74 - protein, 42 aa, Adeno-associated virus 8
PVATEEYGIVADNLQQQNTAPQIGTVNSQGALPGMVWQNRDV

SEQ ID NO: 75 - protein, 42 aa, Adeno-associated virus 9
PVATESYGQVATNHQSAQAQAQTGWVQNQGILPGMVWQDRDV

SEQ ID NO: 76 - protein, 42 aa, Adeno-associated virus 10
PVATEQYGVVADNLQQANTGPIVGNVNSQGALPGMVWQNRDV

(19) United States
(12) Patent Application Publication
(10) Pub. No.: US 2024/0301008 A1
(43) Pub. Date: Sep. 12, 2024

(54) MODIFIED AAV CAPSIDS AND USES THEREOF

(71) Applicant: Adverum Biotechnologies, Inc., Redwood City, CA (US)

(72) Inventor: Annahita KERAVALA, Palo Alto, CA (US)

(73) Assignee: Adverum Biotechnologies, Inc., Redwood City, CA (US)

(21) Appl. No.: 18/678,888

(22) Filed: May 30, 2024

Related U.S. Application Data
(62) Division of application No. 17/514,711, filed on Oct. 29, 2021, now Pat. No. 12,030,914.
Application No. 17/514,711 is a division of application No. 15/788,446, filed on Oct. 19, 2017, now Pat. No. 11,192,925.
(60) Provisional application No. 62/410,291, filed on Oct. 19, 2016.

(51) Int. Cl. C07K 14/005; A61K 9/00; A61K 48/00; C12N 7/00; C12N 15/86

(52) U.S. Cl. CPC C07K 14/005; A61K 9/0048; A61K 48/0008; A61K 48/0075; C12N 7/00; C12N 15/86; C12N 2750/14121; C12N 2750/14122; C12N 2750/14133; C12N 2750/14143; C12N 2750/14171

(57) ABSTRACT

The present disclosure provides adeno-associated virus (AAV) virions with altered capsid protein that binds heparan sulfate proteoglycans, where the AAV virions exhibit greater infectivity of retinal cells, altered tropism and/or the ability to bind and cross the inner limiting membrane following intravitreal injection. The present disclosure further provides methods of delivering a gene product to a retinal cell in an individual, and methods of treating ocular disease.

### CROSS REFERENCE TO RELATED APPLICATIONS

This application is a divisional of U.S. application Ser. No. 17/514,711 filed Oct. 29, 2021; which was a divisional of U.S. application Ser. No. 15/788,446 filed Oct. 19, 2017, now U.S. Pat. No. 11,192,925 issued Dec. 7, 2021; which application claimed priority to and the benefit of U.S. Provisional Application No. 62/410,291, filed on Oct. 19, 2016, and all of which are herein incorporated by reference in their entirety.

### STATEMENT REGARDING SEQUENCE LISTING

The Sequence Listing associated with this application is provided in ST26 XML format and is hereby incorporated by reference into the specification. The name of the xml file containing the Sequence Listing is 17234.019US3.xml. The file was created on May 29, 2024 from the previously submitted ST25 file, and is being submitted electronically.

### FIELD OF THE INVENTION

Embodiments of the present disclosure related to modified viral capsid proteins, including modified AAV capsid proteins, viruses and viral vectors comprising the modified AAV capsid proteins, and methods of using these viruses and viral vectors to deliver polypeptides to cells.

### BACKGROUND OF THE INVENTION

A promising approach to treating and preventing genetic and other diseases and disorders is delivery of therapeutic agents with a gene therapy vector such as a viral vector. Illustrative examples of viral vectors suitable for gene therapy include but are not limited to retroviral vectors, lentiviral vectors, adenovirus vectors, herpes virus vectors, alphavirus vectors, and adeno-associated virus (AAV) vectors. AAV is a 4.7 kb, single-stranded DNA virus. Recombinant vectors based on AAV (rAAV vectors) are associated with excellent clinical safety, since wild-type AAV is nonpathogenic and has no etiologic association with any known diseases. In addition, AAV offers the capability for highly efficient gene delivery and sustained transgene expression in numerous tissues, including eye, muscle, lung and brain. Furthermore, AAV has shown promise in human clinical trials. One example is Leber's congenital amaurosis in which patients treated with a therapeutic delivered by a single subretinal administration of an rAAV vector have experienced sustained clinical benefit from expression of the therapeutic agent for more than four years from the initial date of treatment.

Certain challenges that remain with regard to the design of viral vectors for use in gene therapy include optimizing viral cell tropism and, particularly with respect to gene therapy of the eye, optimizing delivery to the retina. Thus, there is a need for optimized vectors for expressing genes in selected mammalian cells. The present invention addresses this need by providing modified AAV capsid proteins advantageous for the delivery of viral vectors to desired cells and tissues.

### SUMMARY OF THE INVENTION

The present invention relates generally to the field of gene therapy, and in particular, to viral vectors useful for the delivery of nucleic acid segments encoding various agents (e.g., peptides, polypeptides, and polynucleotides, including, e.g., siRNAs, microRNAs, ribozymes, and catalytic RNA molecule), including therapeutic agents, to selected cells and tissues of vertebrate animals. In particular, aspects of the present invention include modified capsid proteins that are useful in gene therapy vectors, including for example, AAV vectors, for the delivery of agents to desired cells and for the treatment of mammalian diseases, disorders, and dysfunctions.

The disclosed compositions may be utilized in a variety of investigative, diagnostic and therapeutic regimens, including the prevention and treatment of a variety of human diseases.

In certain embodiments, the present invention includes a non-naturally-occurring modified AAV2.5T capsid protein, comprising one or more amino acid modifications. In certain embodiments, the modified AAV2.5T capsid protein comprises an amino acid insertion comprising or consisting of an amino acid sequence having at least 80%, at least 85%, or at least 90% homology to the amino acid sequence LALGETTRPA (SEQ ID NO:4), or a fragment of the amino acid sequence comprising at least five, at least six, at least seven, at least eight, or at least nine consecutive amino acids thereof, wherein the modified AAV2.5T capsid protein containing any of the aforesaid insertions is referred to as AAV2.5T/7m8. In certain embodiments, the 7m8 amino acid insertion is located between amino acid residues 574 and 588 of the AAV2.5T capsid protein.

In related embodiments, the present invention provides a modified AAV2.5T or AAV2.5T/7m8 capsid protein, wherein the capsid protein comprises an amino acid modification that introduces one or more amino acids of the AAV2 parental sequence into the capsid protein, and in particular embodiments, wherein the modification confers or enhances heparan sulfate binding to the capsid protein.

In certain aspects, the modified AAV2.5T capsid protein comprises at least one of the one or more of the following amino acid modifications: a S576R point mutation; a T579R point mutation; a substitution of amino acid residues 576-579 or amino acid residues 576-581 with the following amino acid residues; RGNRQA (SEQ ID NO:5); a substitution of amino acid residues 576-581 with the following amino acid residues: RGNRQAAP (SEQ ID NO:6); a substitution of amino acid residues 573-581 or amino acid residues 573-584 with the following amino acid residues: NLQRGNRQAATA (SEQ ID NO:7); or a substitution of amino acid residues 573-581 or amino acid residues 573-584 with the following amino acid residues: NLQRGNRQAATAAP (SEQ ID NO:8).

In particular aspects, the modified AAV2.5T/7m8 capsid protein comprises at least one of the one or more of the following amino acid modifications: a point mutation corresponding to a S576R point mutation in AAV2.5T; a point mutation corresponding to a T579R point mutation in AAV2.5T; a substitution corresponding to a substitution of amino acid residues 576-579 or amino acid residues 576-581 in AAV2.5T with the following amino acid residues: RGNRQA (SEQ ID NO:5); a substitution corresponding to a substitution of amino acid residues 576-579 or amino acid residues 576-581 in AAV2.5T with the amino acid residues: RGNRQAAP (SEQ ID NO:6) or NLQRGNRQAATA (SEQ ID NO:7); a substitution corresponding to a substitution of amino acid residues 573-579, amino acid residues 573-581, amino acid residues 573-583, or amino acid residues 573-584 in AAV2.5T with the following amino acid residues: NLQRGNRQAATA (SEQ ID NO:7); or a substitution corresponding to a substitution of amino acid residues 573-579, amino acid residues 573-581, amino acid residues 573-583, amino acid residues 573-584, or amino acid residues 576-583 in AAV2.5T with the following amino acid residues: NLQRGNRQAATAAP (SEQ ID NO:8).

In certain embodiments, a modified AAV2.5T capsid protein comprises at least one of the one or more of the following amino acid modifications: (a) a S576R point mutation: (b) a T579R point mutation; (c) a substitution of amino acid residues 576-579 or 576-581 with the following amino acid residues: RGNRQA (SEQ ID NO:5); (d) a substitution of amino acid residues 576-581 with the following amino acid residues: RGNRQAAP (SEQ ID NO:6); (e) a substitution of amino acid residues 573-579, 573-581 or 573-584 with the following amino acid residues: NLQRGNRQAATA (SEQ ID NO:7); or (f) a substitution of amino acid residues 573-581 or 573-584 with the following amino acid residues: NLQRGNRQAATAAP (SEQ ID NO:8).

In certain embodiments, a modified AAV2.5T/7m8 capsid protein comprises at least one or more of the following amino acid modifications: (a) a point mutation corresponding to a S576R point mutation in AAV2.5T; (b) a point mutation corresponding to a T579R point mutation in AAV2.5T; (c) a substitution corresponding to a substitution of amino acid residues 576-579 or 576-581 in AAV2.5T with the following amino acid residues: RGNRQA (SEQ ID NO:5); (d) a substitution corresponding to a substitution of amino acid residues 576-581 in AAV2.5T with the following amino acid residues: RGNRQAAP (SEQ ID NO:6); (e) a substitution corresponding to a substitution of amino acid residues 573-579, 573-581, 573-583, 573-584, 576-579, 576-581, 576-583 or 576-584 in AAV2.5T with the following amino acid residues: NLQRGNRQAATA (SEQ ID NO:7); or (f) a substitution corresponding to a substitution of amino acid residues 576-583, 576-584, 573-583 or 573-584 in AAV2.5T with the following amino acid residues: NLQRGNRQAATAAP (SEQ ID NO:8).

In particular embodiments, the modified capsid protein comprises one or modifications shown in any of the accompanying figures. In certain embodiments, the modified AAV2.5T/7m8 capsid protein is an AAV2.5T/7m8(0)), AAV2.5T/7m8(+3), AAV2.5T/7m8(−3), or AAV2.5T/7m8(−12). In particular embodiments, the modified capsid protein comprises a sequence shown in FIG. 4A, FIG. 4B or FIG. 4C or set forth in any of SEQ ID NOs:1-3.

In a related embodiment, the present invention includes a polynucleotide comprising a nucleic acid sequence encoding a modified AAV2.5T or AAV2.5T/7m8 capsid protein described herein. In certain embodiments, the nucleic acid sequence encoding the modified AAV2.5 or AAV2.5T capsid protein is operably linked to a promoter sequence. In particular embodiments, the polynucleotide further comprises a nucleic acid sequence encoding a rep protein.

A further related embodiment of the present invention includes a cell comprising an expression vector described herein. In further embodiments, the cell comprises a polynucleotide that encodes a therapeutic protein.

Another embodiment is a recombinant virus or viral vector comprising a modified capsid protein described herein. In certain embodiments, the recombinant virus or viral vector is an adeno-associated virus (AAV), optionally AAV2. In particular embodiments, the recombinant virus or viral vector is eluted from a heparan column at a salt concentration of about 0.2 M to about 0.4 M. In some embodiments, the recombinant virus or viral vector is capable of binding to and crossing the inner limiting membrane (ILM) when intravitreally injected into a mammal. In certain embodiments, the recombinant virus or viral vector comprises a polynucleotide sequence that encodes a therapeutic agent. In certain embodiments, the therapeutic agent is a therapeutic protein. In other embodiments, the therapeutic agent is a therapeutic nucleic acid. Exemplary therapeutic nucleic acids include siRNA, shRNA, and ribozymes. In particular embodiments, the therapeutic protein is an anti-vascular endothelial growth factor (anti-VEGF) agent. Exemplary anti-VEGF agents include aflibercept, ranibizumab and bevacizumab.

In certain embodiments, a recombinant virus or viral vector described herein has an altered cellular tropism as compared to AAV2.5T or AAV2.5T/7m8. In some embodiments, the recombinant virus or viral vector is a AAV2.5T comprising a modified capsid protein comprising: (a) a S576R point mutation; or (b) a T579R point mutation, wherein the recombinant virus or viral vector has a greater tropism for retinal ganglion cells (“RGC”) than AAV2.5T. In other embodiments, the recombinant virus or viral vector is a AAV2.5T/7m8 comprising a modified capsid protein comprising: (a) a S576R point mutation; and (b) a T579R point mutation, wherein the recombinant virus or viral vector has a greater tropism for Müller cells than AAV2.5T/7m8. In other embodiments, the recombinant virus or viral vector is a AAV2.5T/7m8 comprising a modified capsid protein comprising a substitution of amino acid residues corresponding to 573-584 in AAV2.5T/7m8 with the following amino acid residues: NLQRGNRQAATA (SEQ ID NO:7), wherein the recombinant virus or viral vector has a greater tropism for retinal cells than AAV2.5T/7m8.

In further related embodiments, the present invention includes a pharmaceutical composition comprising a recombinant virus or viral vector described herein.

The present invention also includes a related method of providing a protein to a retina of a subject, comprising administering to the subject by intravitreal injection the recombinant virus or viral vector or pharmaceutical composition described herein, wherein the recombinant virus or viral vector comprises a polynucleotide sequence that encodes the protein.

The present invention further includes a method of providing a therapeutic gene product (e.g., a therapeutic protein) to a retina of a subject in need thereof, comprising administering to the subject by intravitreal injection a pharmaceutical composition comprising a recombinant virus or viral vector described herein, wherein the recombinant virus or viral vector comprises a polynucleotide encoding the therapeutic gene product. In particular embodiments, subject has been diagnosed with or is considered at risk of an ocular disease or disorder. In particular embodiments, the subject has been diagnosed with or is suspected of having or being at risk of developing one or more conditions selected from the group consisting of: age-related macular degeneration (AMD), wet-AMD, dry-AMD, retinal neovascularization, choroidal neovascularization, diabetic retinopathy, proliferative diabetic retinopathy, retinal vein occlusion, central retinal vein occlusion, branched retinal vein occlusion, diabetic macular edema, diabetic retinal ischemia, ischemic retinopathy, and diabetic retinal edema.

The present invention further provides a method of altering the tropism of an AAV2.5T or AAV2.5T/7m8 virus or viral vector, comprising introducing one or more amino acid modifications that confers or increases heparan sulfate binding to a capsid protein of the virus or viral vector. In particular embodiments, the amino acid modification introduces one or more amino acids of AAV2 into the capsid protein. In particular embodiments, at least one of the one or more amino acid modifications comprises: a point mutation corresponding to a S576R point mutation in AAV2.5T; a point mutation corresponding to a T579R point mutation in AAV2.5T; a substitution corresponding to a substitution of amino acid residues 576-581 in AAV2.5T with amino acid residues: RGNRQA (SEQ ID NO:5) or RGNRQAAP (SEQ ID NO:6); or a substitution corresponding to a substitution of amino acid residues 573-581, 576-579, 573-583 in AAV2.5T with amino acid residues: NLQRGNRQAATA (SEQ ID NO:7) or NLQRGNRQAATAAP (SEQ ID NO:8). In one embodiment, the amino acid modification comprises: (a) a S576R point mutation; or (b) a T579R point mutation, wherein the virus or viral vector has a greater tropism for RGC than AAV2.5T. In one embodiment, the amino acid modification comprises: (a) a S576R point mutation; and (b) a T579R point mutation, wherein the virus or viral vector has a greater tropism for Müller cells than AAV2.5T/7m8. In one embodiment, the amino acid modification comprises a substitution of amino acid residues corresponding to 576-579 in AAV2.5T with the following amino acid residues: NLQRGNRQAATA (SEQ ID NO:7), wherein the recombinant virus or viral vector has a greater tropism for retinal cells than AAV2.5T/7m8. In particular embodiment, the recombinant virus or viral vector is AAV2.5T/7m8−12-IHSPG(extra8) comprising a modified capsid sequence shown in FIG. 3C.

In another embodiment, the present invention includes a method for selectively delivering a polypeptide to RGC of a subject, comprising administering to the subject a modified AAV2.5T virus or viral vector comprising a polynucleotide that encodes the polypeptide, wherein the virus or viral vector comprises a capsid protein comprising a S576R point mutation or a T579R point mutation.

In another embodiment, the present invention includes a method for selectively delivering a polypeptide to Müller cells of a subject, comprising administering to the subject a modified AAV2.5T/7m8 virus or viral vector comprising a polynucleotide that encodes the polypeptide, where the virus comprises a capsid protein comprising: (a) a S576R point mutation; and (b) a T579R point mutation, optionally AAV2.5T/7m8(+3)-2pt.

In another embodiments, the present invention includes a method for selectively delivering a polypeptide to retinal cells of a subject, comprising administering to the subject a modified AAV2.5T/7m8 virus or viral vector comprising a polynucleotide that encodes the polypeptide, where the virus comprises a capsid protein comprising a substitution of amino acid residues corresponding to amino acid residues 576-579 in AAV2.5T with amino acid residues: NLQRGNRQAATA (SEQ ID NO:7) or NLQRGNRQAATAAP (SEQ ID NO:8), or a substitution of amino acid residues corresponding to amino acid residues 573-579 in AAV2.5T with amino acid residues: TNQNLQRGNRQAATA (SEQ ID NO:9) or TNQNLQRGNRQAATAAP (SEQ ID NO:10).

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1A provides fundus images (top panels) and immunofluorescent stains (bottom panels) of retina following sub-retinal (left panels) or intravitreal injection (right panels) of AAV2.5T virus encoding green fluorescent protein (GFP). GFP expression following sub-retinal injection colocalized with rhodopsin expression, indicating that photoreceptors were most effectively transduced, further, GFP expression was significantly greater than expression after intravitreal injection, which demonstrate that AAV2.5T efficiently transduces photoreceptors when injected sub-retinally, but is unable to efficiently transduce the outer retina when intravitreally injected.

FIG. 1B provides graphs showing the results of in vitro IVIg assays, which demonstrate that AAV2.5T has a better neutralizing antibody (nAb) profile compare to AAV2 as evidenced by its reduced IC₅₀.

FIG. 2 is a diagram showing amino acid residues 580-594 of the AAV2 capsid protein (SEQ ID NO:44), and indicating the amino acid residues modified by various point mutations and swaps in illustrative modified capsid proteins described herein.

FIG. 3A provides a sequence alignment of the HSPG binding region of the indicated capsids (SEQ ID NOs: 44-53). Sequences from the parental AAV2 sequence are indicated in grey. AAV2.5T-2pt (SEQ ID NO:49) includes the indicated point mutations at amino acid residues 576 and 579. AAV2.5T-sHSPG(correct) (SEQ ID NO:50) includes a replacement of amino acid residues 576-581 of AAV2.5 by amino acid residues 585-590 of AAV2. AAV2.5T-sHSPG(extra2) (SEQ ID NO:51) includes a replacement of amino acid residues 576-580 of AAV2.5 by amino acid residues 585-590 of AAV2. resulting in an increase in total length of 2 amino acids. AAV2.5T-IHSPG(correct) (SEQ ID NO:52) includes a replacement of amino acid residues 573-584 of AAV2.5T by the amino acid residues 582-593 of AAV2. AAV2.5T-IHSPG(extra8) (SEQ ID NO:53) includes a replacement of amino acid residues 576-579 of AAV2.5T by the amino acid residues 582-593 of AAV2. resulting in an increase in the total length of 8 amino acids.

FIG. 3B provides a sequence alignment of the HSPG binding region of indicated capsids (SEQ ID NOs: 4. 46 and 54-68). Sequences from the parental AAV2 sequence are indicated in light grey. and the 7m8 sequence (LALGETTRPA: SEQ ID NO:4) is indicated in dark grey. AAV2.5T/7m8 (SEQ ID NOs: 55-68) includes a 10 amino acid insertion. LALGETTRPA (SEQ ID NO:4). as compared to 2.5T (SEQ ID NO:46). The location of the insertion is indicated by the parenthetical following “7m8” in the various versions of 2.5T/7m8 (e.g., 2.5T/7m8(+3) (SEQ ID NO:56). 2.5T/7m8(0)) (SEQ ID NO:55). In AAV2.5T/7m8. the 7m8 sequence is inserted between T578 and T579 of the AAV2.5T sequence. which is equivalent to the location of the 7m8 insertion in AAV2/7m8 (SEQ ID NO:54). AAV2.5T/7m8(+3) (SEQ ID NO:56) had the 7m8 insertion shifted one residue towards the C-terminus (+3 indicating the shift 3 nucleotides in the DNA sequence used to produce the recombinant capsid); the capsids designated “ . . . 7m8(−3)”; “ . . . 7m8(−6)”; “ . . . 7m8(−9)”: and “ . . . 7m8(−12)” are shifted 1, 2, 3, and 4 amino acids, respectively, toward the N-terminus. 2.5T/7m8(+3)-2pt (SEQ ID NO:61) includes the indicated point mutations to arginine at amino acid residues 576 and 579: 2.5T/7m8(0)-sHSPG (SEQ ID NO:63) includes a replacement of amino acid residues 576-591 by the indicated HSPG binding loop of AAV2. which also includes the 7m8 10 amino acid insertion within the HSPG binding loop; and 2.5T/7m8(0))-IHSPG (SEQ ID NO:65) includes a replacement of amino acid residues 573-591 by the indicated longer version of the HSPG binding loop of AAV2, which also includes the 7m8 10 amino acid insertion within the HSPG binding loop. 2.5T/7m8−12-sHSPG(extra2) (SEQ ID NO:67) includes a replacement of amino acid residues 576-579 by the indicated HSPG binding loop of AAV2: 2.5T/7m8−12-IHSPG(extra8) (SEQ ID NO:68) includes a replacement of amino acid residues 576-579 by the indicated HSPG binding loop of AAV2: 2.5T/7m8−12-sHSPG(correct) includes a replacement of amino acid residues 575-581 of 2.5T by the indicated HSPG binding loop of AAV2: and 2.5T/7m8−12-IHSPG(correct) includes a replacement of amino acid residues 573-581 of AAV2.5T by the indicated longer version of the HSPG binding loop of AAV2, which also includes the 7m8 10 amino acid insertion within the HSPG binding loop.

FIG. 4A provides the amino acid sequence of the capsid protein of AAV2.5T/7m8(+3)Δ2 (also referred to as 2.5T/7m8(+3)-2pt) (SEQ ID NO:1).

FIG. 4B provides the amino acid sequence of the capsid protein of AAV2.5T/7m8(−12) elHSPG (also referred to as 2.5T/7m8(−12)-IHSPG(extra8)) (SEQ ID NO:2).

FIG. 4C provides the amino acid sequence of the capsid protein of AAV2.5T/7m8(−3) IHSPG (SEQ ID NO:3).

FIG. 5 shows the dot blot results of HSPG column fractions from different 2.5 swap variants. Elutes E1 to E10 have increasing concentrations of NaCl of 0.1M (E1), 0.2M (E2), 0.3M(E3), 0.4M (E4), 0.5M (E5), 0.6M (E6), 0.7M (E7), 0.8M (E8), 0.9M (E9) and 1.0M (E10).

FIG. 6 shows live fluorescent images of GFP expression in HEK293 cells transduced with the indicated HSPG swap variants. The percentage of HEK293 cells expressing GFP is indicated in parenthesis above the image.

FIGS. 7A-7K show live images of GFP expression in pig retinal explants two weeks following infection with the indicated AAV vector expressing GFP. FIG. 7A shows GFP expression using the 2.5T vector; FIG. 7B shows GFP expression using the 2.5T/7m8(−3) vector: FIG. 7C shows GFP expression using the 2.5T-S576R vector; FIG. 7D shows GFP expression using the 2.5T-T579R vector; FIG. 7E shows GFP expression using the 2.5T/7m8(+3)-2pt vector; FIG. 7F shows GFP expression using the 2.5T/7m8(0))-sHSPG vector; FIG. 7G shows GFP expression using the 2.5T/7m8(−12)-2pt vector; FIG. 7H shows GFP expression using the 2.5T/7m8(−12)-sHSPG-extra2 vector; FIG. 7I shows GFP expression using the 2.5T/7m8(−12)-IHSPG-extra8 vector; FIG. 7J shows GFP expression using the 2.5T/7m8(−12)-IHSPG-correct vector; and FIG. 7K shows GFP expression using the 2.5T/7m8(−12)-sHSPG-correct vector.

FIGS. 8A-8F provide immunofluorescence images of GFP expression in retinal cells of cryosectioned pig explants two weeks following infection with the indicated AAV viruses expressing GFP. In the immunofluorescence images, the cells are stained with DAPI, and probed with antibodies to detect the following proteins: Rho (to detect rod cells), Tuj1 (to detect RGC), GFAP (to detect Müller cells) and CX10) (to detect bipolar cells). In each pair of images, the left panel shows overlap of the stains, and the right panel shows GFP single channel staining. FIG. 8A shows cells infected with the 2.5T parent vector; FIG. 8B shows cells infected with the 2.5T-S576R vector; FIG. 8C shows cells infected with the 2.5T-T579R vector; FIG. 8D shows cells infected with the 2.5T/7m8(−3) vector; FIG. 8E shows cells infected with the 2.5T/7m8(+3)-2pt vector; and FIG. 8F shows cells transfected with the 2.5T/7m8(−12)-IHSPG-extra8 vector.

FIG. 9 shows brightfield images (top panels) and fluorescence fundus images (lower panels) of gerbil retina eight weeks following intravitreal administration of the indicated viruses expressing GFP.

FIG. 10 provides amino acid sequences corresponding to amino acids 570-610 of AAV2 of AAV capsid protein VP1 of various AAV serotypes (SEQ ID NOs: 69-76).

FIGS. 11A-11E provide graphs showing the results of in vitro IVIg assays, which demonstrate that the indicated viruses have safe nAb profiles, similar to the parent 2.5T, as evidenced by the similarly low IC₅₀ values, and significantly lower than the C50 value of AAV2 (see FIG. 1B top panel). FIG. 11A show results from the 2.5T parent virus; FIG. 11B shows results from the 2.5T/7m8 (control) virus; FIG. 11C shows results from the 2.5T/7m8(+3) Δ2 virus; FIG. 11D shows results from the 2.5T/7m8(−12) elHSPG virus; and FIG. 11E shows results from the 2.5T/7m8(−3) sHSPG virus.

FIG. 12A and FIG. 12B provide immunofluorescence images of GFP expression in gerbil retina eight weeks following intravitreal injections of AAV2.5T/7m8−12 elHSPG (also referred to as AAV2.5T/7m8(−12)-IHSPG(extra8)) expressing GFP. In the immunofluorescence images, the cells are stained with DAPI, and also probed with antibodies to L/M Opsin (FIG. 12A), or stained with DAPI, and probed to detect parvalbumin, and rhodopsin (FIG. 12B). Fluorescence patterns that extend evenly across the transverse section are indicated in the margin of each immunofluorescence image. GFP expression colocalized with parvalbumin and DAPI staining.

FIG. 13 provides an immunofluorescence image of GFP expression in gerbil retina eight weeks following intravitreal injection of AAV2.5T/7m8−3 IHSPG expressing GFP. In the immunofluorescence image the cells are stained with DAPI, and probed to detect parvalbumin. and rhodopsin. Fluorescence patterns that extend evenly across the transverse section are indicated in the margin of each immunofluorescence image. White arrowheads indicate to points of GFP expression where expression is not uniform across a cell layer in the immunofluorescence image. GFP expression colocalized primarily with parvalbumin and DAPI.

FIG. 14A and FIG. 14B provide immunofluorescence images of GFP expression in gerbil retina eight weeks following intravitreal injection of AAV2.5T/7m8(+3)Δ2 expressing GFP. In the top immunofluorescence image (FIG. 14A), the cells are stained with DAPI, and probed to detect parvalbumin. and rhodopsin. Fluorescence patterns that extend evenly across the transverse section are indicated in the margin of each immunofluorescence image. White arrowheads indicate to points of GFP expression where expression is not uniform across a cell layer in the immunofluorescence image. In the bottom immunofluorescence image (FIG. 14B), the cells are stained with DAPI, and probed to detect GFP and L/M opsin.

FIG. 15 provides an OTC autofluorescence image of the retina of a green monkey twelve weeks after intravitreal administration of 2.5E+12 vg/eye of AAV 2.5T/7m8(−12) elHSPG. Robust GFP expression can be observed in the macula (indicated by the black arrow) as well as inside the blood arcades (indicated by the white arrow). GFP expression

FIG. 16 provides a live fluorescence image of a flat-mounted green monkey retina extracted twelve weeks following intravitreal administration of 2.5E+12 vg/eye of AAV 2.5T/7m8(−12) elHSPG. GFP expression is present in the macula. as well as within the blood arcades. Arrows indicate the location of the transverse sections depicted in FIGS. 17A-17C.

FIGS. 17A-17C provide immunofluorescence images of GFP expression in retinal cells of a cryosectioned green monkey retina twelve weeks following intravitreal administration of 2.5E+12 vg/eye of AAV 2.5T/7m8(−12) elHSPG. Sections were taken in the fovea (FIG. 17A), mid-periphery (FIG. 17B). and periphery (FIG. 17C). In FIG. 17A. a section of the fovea. the cells are stained with DAPI, and probed to detect GFP. Calbindin (bipolar cells), and S-opsin (S Cones). In FIG. 17B, a cross section of the mid-periphery of the retina, the cells are stained with DAPI, and probed to detect GFP, rhodopsin (rods), and L/M-opsin (L/M cones). In FIG. 17C, a cross section of the periphery of the retina, cells are stained with DAPI, and probed to detect GFP, glutamine synthetase (Müller cells), and L/M-opsin (L/M Cones). GFP expression is present in all three figures. GFP colocalizes with RGCs, cones, and Müller cells and is robustly expressed.

FIG. 18 provides an immunofluorescence image of GFP expression in a cross section of the temporal retinal petal of a green monkey. twelve weeks after intravitreal administration of 2.5E+12 vg/eye of AAV2.5T/7m8(−12) elHSPG. GFP expression is present from the fovea (marked A) to the or a serrata (marked B).

FIG. 19 provides an OTC autofluorescence image of a green monkey retina twelve weeks following intravitreal administration of 2.5E+12 vg/eye of AAV2.5T/7m8(+3)Δ2. Robust GFP expression can be observed in the macula (indicated by the white arrow).

FIG. 20 provides a live fluorescence image of a flat-mounted green monkey retina extracted twelve weeks following intravitreal administration of 2.5E+12 vg/eye of AAV2.5T/7m8(+3)42. Arrow indicates the location of the transverse section depicted in FIG. 21. Expression GFP appears primarily in the macula.

FIG. 21 provides an immunofluorescence image of GFP expression in retinal cells of a cryosectioned green monkey retina twelve weeks after intravitreal administration of 2.5E+12 vg/eye of AAV2.5T/7m8(+3)42. The sections was taken in the fovea. In the immunofluorescence images, the cells are stained with DAPI, and probed to detect GFP, PNA (cones), and vimentin (Müller cells). GFP expression colocalizes with vimentin in the fovea, indicating that transduction occurred primarily in this region.

### DETAILED DESCRIPTION OF THE INVENTION

The present disclosure provides modified capsid proteins and virions and viral vectors having one or more modified or altered capsid protein. where in various embodiments, the virions exhibit: 1) increased infectivity of a retinal cell; 2) altered tropism; 3) increased binding to heparan or heparan sulfate proteoglycans and/or the inner limiting membrane (ILM); and/or 4) an increased ability to infect and/or deliver a therapeutic gene product across the ILM when administered intravitreally, as compared to a corresponding virion comprising its native or wild-type capsid protein instead of a modified capsid protein disclosed herein. Also provided are pharmaceutical compositions and methods for the use of any of the compositions disclosed herein for promoting the expression of a gene in cells, e.g., retinal cells. in an individual, e.g., for the treatment or prophylaxis of a disease or disorder. These and other objects, advantages, and features of the invention will become apparent to those persons skilled in the art upon reading the details of the compositions and methods as more fully described below.

### DEFINITIONS

A “vector” as used herein refers to a macromolecule or association of

macromolecules that comprises or associates with a polynucleotide and which can be used to mediate delivery of the polynucleotide to a cell. Illustrative vectors include, for example, plasmids, viral vectors, liposomes, and other gene delivery vehicles.

The term “AAV” is an abbreviation for adeno-associated virus, and may be used to refer to the virus itself or derivatives thereof. The term covers all subtypes and both naturally occurring and recombinant forms, except where required otherwise. The term “AAV” includes AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

The terms “AAV2.5T” and “2.5T” are used interchangeably, as are the terms “AAV2.5T/7m8” and “2.5T/7m8”. The term “IHSPG(extra8)” and the term “elHSPG” refer to the same sequence and are used interchangeably. The term “2pt” and “Δ2” are used interchangeably, and refer to capsids containing both the S576R and T579R mutations in the AAV2.5T capsid.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077 (AAV-1), AF063497 (AAV-1), NC_001401 (AAV-2), AF043303 (AAV-2), NC_001729 (AAV-3), NC _-001829 (AAV-4), U89790 (AAV-4), NC_006152 (AAV-5), AF513851 (AAV-7), AF513852 (AAV-8), and NC_006261 (AAV-8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99:11854; Moris et al. (2004) Virology 33:375-383; international patent publications WO 00/28061, WO 99/61601, WO 98/11244; and U.S. Pat. No. 6,156,303.

An “AAV virus” or “AAV viral particle” or “TAAV vector particle” or “rAAV particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome such as a transgene to be delivered to a mammalian cell), it is typically referred to as a “rAAV vector particle” or simply a “rAAV vector”. Thus, production of rAAV particle necessarily includes production of rAAV vector, as such a vector is contained within a rAAV particle.

The term “replication defective” as used herein relative to an AAV viral vector of the invention means the AAV vector cannot independently replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate further.

An “AAV variant” or “AAV mutant” as used herein refers to a viral particle composed of: a) a variant AAV capsid protein, where the variant AAV capsid protein comprises at least one amino acid difference (e.g., amino acid substitution, amino acid insertion, amino acid deletion) relative to a corresponding parental AAV capsid protein, where the AAV capsid protein does not correspond to the amino acid sequence present of a naturally occurring AAV capsid protein; and, optionally, b) a heterologous nucleic acid comprising a nucleotide sequence encoding a heterologous gene product, wherein the variant AAV capsid protein confers increased binding to heparan or a heparan sulfate proteoglycan as compared to the binding by an AAV virion comprising the corresponding parental AAV capsid protein. In certain embodiments, the variant capsid protein confers: a) increased infectivity of a retinal cell compared to the infectivity of the retinal cell by an AAV virion comprising the corresponding parental AAV capsid protein; b) altered cellular tropism as compared to the tropism of an AAV virion comprising the corresponding parental AAV capsid protein; and/or c) an increased ability to bind and/or cross the ILM as compared to an AAV virion comprising the corresponding parental AAV capsid protein.

The abbreviation “rAAV” refers to recombinant adeno-associated virus, also referred to as a recombinant AAV vector (or “rAAV vector”). A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans. For example, a plasmid or other expression vector comprising nucleotide sequences encoding one or more adenoviral proteins is transfected into a producer cell along with an TAAV vector.

An “infectious” virus or viral particle is one that comprises a competently assembled viral capsid and is capable of delivering a polynucleotide component into a cell for which the viral species is tropic. The term does not necessarily imply any replication capacity of the virus. Assays for counting infectious viral particles are described elsewhere in this disclosure and in the art. Viral infectivity can be expressed as the ratio of infectious viral particles to total viral particles. Methods of determining the ratio of infectious viral particle to total viral particle are known in the art. See, e.g., Grainger et al. (2005) Mol. Ther. 11:S337 (describing a TCID50 infectious titer assay); and Zolotukhin et al. (1999) Gene Ther. 6:973. See also the Examples.

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The term “polynucleotide” refers to a polymeric form of nucleotides of any length. including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double-and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970)

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters:
Mismatch Penalty: 1.00; Gap Penalty: 1.00; Gap Size Penalty: 0.33; and Joining Penalty: 30.0.

A “gene” refers to a polynucleotide containing at least one open reading frame that is capable of encoding a particular gene product after being transcribed, and sometimes also translated. The term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence. For example, the gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

A “gene product” is a molecule resulting from expression of a particular gene. Gene products include, e.g., a polypeptide, an aptamer, an interfering RNA, an mRNA, and the like. In particular embodiments, a “gene product” is a polypeptide, peptide, protein or interfering RNA including short interfering RNA (siRNA), miRNA or small hairpin RNA (shRNA). In particular embodiments, a gene product is a therapeutic gene product, e.g., a therapeutic protein.

As used herein, a “therapeutic gene” refers to a gene that, when expressed, produces a therapeutic gene product that confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include, but are not limited to, genes that correct a genetic deficiency in a cell or mammal.

As used herein, a “transgene” is a gene that is delivered to a cell by a vector.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a gene product of interest, and is used for effecting the expression of the gene product in an intended target cell. An expression vector also comprises control elements operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

As used herein, the terms “polypeptide,” “peptide,” and “protein” refer to polymers of amino acids of any length. The terms also encompass an amino acid polymer that has been modified; for example, disulfide bond formation, glycosylation, lipidation, phosphorylation, or conjugation with a labeling component.

By “comprising” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g. poly-adenylation sequence, enhancer elements, other genes, linker domains, etc.

By “consisting essentially of”, it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g. linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g. 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of”, it is meant the exclusion from the composition, method, or kit of any element, step, or ingredient not specified in the claim. For example, an expression cassette “consisting of” a gene encoding a therapeutic polypeptide operably linked to a promoter, and a polyadenylation sequence consists only of the promoter, polynucleotide sequence encoding the therapeutic polypeptide, and polyadenlyation sequence. As another example, a polypeptide “consisting of” a recited sequence contains only the recited sequence.

An “expression vector” as used herein encompasses a vector, e.g. plasmid, minicircle, viral vector, liposome, and the like as discussed above or as known in the art, comprising a polynucleotide which encodes a gene product of interest, and is used for effecting the expression of a gene product in an intended target cell. An expression vector also comprises control elements operatively linked to the encoding region to facilitate expression of the gene product in the target. The combination of control elements, e.g. promoters, enhancers, UTRs, miRNA targeting sequences, etc., and a gene or genes to which they are operably linked for expression is sometimes referred to as an “expression cassette.” Many such control elements are known and available in the art or can be readily constructed from components that are available in the art.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

An “enhancer” as used herein encompasses a cis-acting element that stimulates or inhibits transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “termination signal sequence” as used herein encompasses any genetic element that causes RNA polymerase to terminate transcription, such as for example a polyadenylation signal sequence.

A “polyadenylation signal sequence” as used herein encompasses a recognition region necessary for endonuclease cleavage of an RNA transcript that is followed by the polyadenylation consensus sequence AATAAA. A polyadenylation signal sequence provides a “poly A site”, i.e. a site on a RNA transcript to which adenine residues will be added by post-transcriptional polyadenylation.

As used herein, the terms “operatively linked” or “operably linked” refers to a juxtaposition of genetic elements, e.g. promoter, enhancer, termination signal sequence, polyadenylation sequence, etc., wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained. As used herein, the term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. As another example, a promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

The term “endogenous” as used herein with reference to a nucleotide molecule or gene product refers to a nucleic acid sequence, e.g. gene or genetic element, or gene product, e.g. RNA, protein, that is naturally occurring in or associated with a host virus or cell.

The term “native” as used herein refers to a nucleotide sequence, e.g. gene, or gene product, e.g. RNA, protein, that is present in a wildtype virus or cell. The term “variant” as used herein refers to a mutant of a reference polynucleotide or polypeptide sequence, for example a native polynucleotide or polypeptide sequence, i.e. having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence. e.g. a native polynucleotide or polypeptide sequence. For example, a variant may be a polynucleotide having a sequence identity of 70% or more with a full length native polynucleotide sequence, e.g. an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g. an identity of 75% or 80% or more, such as 85%, 90%, or 95% or more, for example, 98% or 99% identity with the full length native polypeptide sequence. Variants may also include variant fragments of a reference, e.g. native, sequence sharing a sequence identity of 70% or more with a fragment of the reference, e.g. native, sequence, e.g. an identity of 75% or 80% or more, such as 85%, 90%, or 95% or more, for example, 98% or 99% identity with the native sequence.

As used herein, the terms “biological activity” and “biologically active” refer to the activity attributed to a particular biological element in a cell. For example, the “biological activity” of an “immunoglobulin”, “antibody” or fragment or variant thereof refers to the ability to bind an antigenic determinant and thereby facilitate immunological function. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g. promoter, enhancer, kozak sequence, and the like, refers to the ability of the regulatory element or functional fragment or variant thereof to regulate, i.e. promote, enhance, or activate the translation of, respectively, the expression of the gene to which it is operably linked.

The terms “administering” or “introducing”, as used herein, refer to delivery of a vector for recombinant gene or protein expression to a cell, to cells and/or organs of a subject, or to a subject. Such administering or introducing may take place in vivo, in vitro or ex vivo. A vector for expression of a gene product may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus: or transduction, which typically means stable infection of a cell with a virus or the transfer of genetic material from one microorganism to another by way of a viral agent (e.g., a bacteriophage).

“Transformation” is typically used to refer to bacteria comprising heterologous DNA or cells which express an oncogene and have therefore been converted into a continuous growth mode such as tumor cells. A vector used to “transform” a cell may be a plasmid, virus or other vehicle.

Typically, a cell is referred to as “transduced”, “infected”: “transfected” or

“transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

The terms “treatment”, “treating” and the like are used herein to generally mean obtaining a desired pharmacologic and/or physiologic effect. The effect may be prophylactic in terms of completely or partially preventing a disease or symptom thereof, e.g. reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

The various compositions and methods of the invention are described below. Although particular compositions and methods are exemplified herein, it is understood that any of a number of alternative compositions and methods are applicable and suitable for use in practicing the invention. It will also be understood that an evaluation of the expression constructs and methods of the invention may be carried out using procedures standard in the art.

The practice of the present invention will employ, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing-herein is to be construed as an admission that the present invention is not entitled to antedate such publication by virtue of prior invention. Further, the dates of publication provided may be different from the actual publication dates which may need to be independently confirmed.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### Variant AAV Capsid Polypeptides

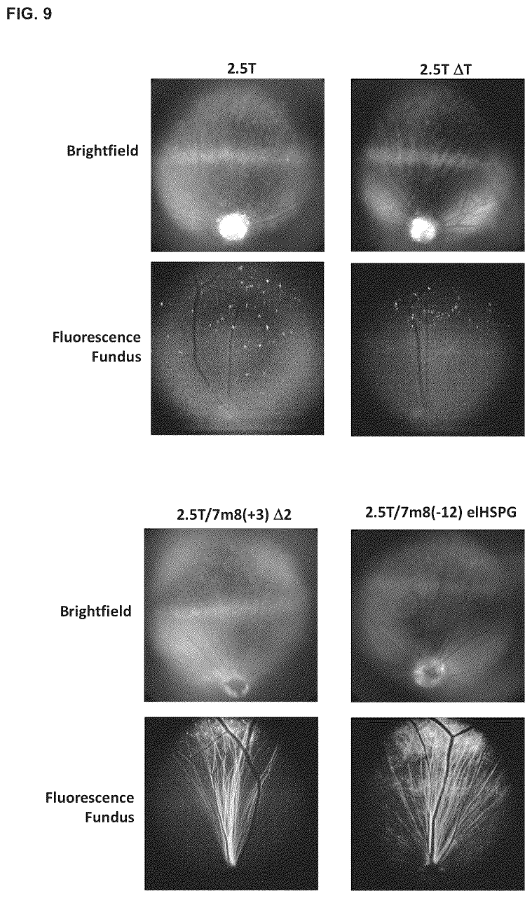

The present disclosure provides variant AAV capsid proteins, where the variant AAV capsid protein comprises one or more amino acid modifications as compared to the wild-type AAV or a corresponding parental AAV. In particular embodiments, the variant AAV capsid protein comprises one or more amino acid modifications as compared to a capsid protein of AAV2.5T, AAV7m8, or AAV2.5T/7m8. In particular embodiments, the variant AAV capsid protein comprises one or more amino acid modifications as compared to a capsid protein having at least 90%, at least 95%, at least 98%, or at least 99% sequence identity to an AAV2.5T, AAV7m8, or AAV2.5T/7m8 capsid protein. In particular embodiments, the variant AAV capsid protein comprises one or more amino acid modifications as compared to a capsid protein of AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV. equine AAV, primate AAV, non-primate AAV, or ovine AAV. In particular embodiments, the variant AAV capsid protein comprises one or more amino acid modifications as compared to a capsid protein having at least 90%, at least 95%, at least 98%, or at least 99% sequence identity to a capsid protein of AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, or ovine AAV. While reference is made herein to amino acid modifications of capsid proteins (including specific amino acid substitutions and insertions) using the amino acid numbering corresponding to AAV2 or AAV2.5T capsid protein, it is understood that any of these amino acid modifications may also be introduced in the capsid protein of AAVs of other serotypes, e.g., at positions corresponding to those of AAV2 or AAV2.5T. FIG. 10 provides an alignment of amino acids corresponding to 570-610 of AAV2 for various AAV serotypes. These sequences share significant homology and similar amino acid numbering, and the skilled artisan can readily determine amino acid residues in other AAV serotypes that correspond to those specifically described herein for AAV2 or AAV2.5T.

In particular embodiments, the variant capsid protein, when present in an AAV virion, confers increased infectivity of a retinal cell as compared to the infectivity of the retinal cell by an AAV virion comprising the corresponding parental AAV capsid protein. In some cases, the retinal cell is a photoreceptor cell (e.g., rods; cones). In other cases, the retinal cell is an RGC. In other cases, the retinal cell is an RPE cell. In other cases, the retinal cell is a Müller cell. Other retinal cells include amacrine cells, bipolar cells, and horizontal cells. In particular embodiments, the variant capsid protein, when present in an AAV virion, confers altered tropism as compared to the tropism of the retinal cell by an AAV virion comprising the corresponding parental AAV capsid protein. In particular embodiments, the variant capsid protein, when present in an AAV virion, confers increased binding to heparan or heparan sulfate, and/or increased ability to bind and cross the inner limiting membrane following intravitreal injection, as compared to an AAV virion comprising the corresponding parental AAV capsid protein.

In certain embodiments, the parental AAV capsid protein is an AAV2.5T capsid protein, or a variant thereof having at least 90%, at least 95%, at least 98%, at least 99% sequence identity to an AAV2.5T capsid protein. AAV2.5T capsid proteins and virions are described in U.S. Pat. No. 9,233,131, in which the VP1-encoding amino acid sequences of AAV2.5T is provided as SEQ ID NO:42 and FIGS. 10A-B. An AAV2.5T VP-1 amino acid sequence is also provided herein as SEQ ID NO:41.

In certain embodiments, the parental AAV capsid protein is an AAV7m8 capsid protein, or a variant thereof having at least 90%, at least 95%, at least 98%, at least 99% sequence identity to an AAV7m8 capsid protein. AAV7m8 capsid proteins are described in U.S. Pat. No. 9,193,956. AAV7m8 includes an at least five amino acid insert (the “7m8 insert”) within the capsid protein of AAV2, located between amino acids 587 and 588 of the wildtype AAV2 genome. In certain embodiment, the 7m8 amino acid insert comprises or consists of the following amino acid sequence: LGETTRP (SEQ ID NO:11). In particular embodiments, the 7m8 insert comprises or consists of the amino acid sequence: LALGETTRPA (SEQ ID NO:4), or a fragment comprising at least five, at least six, at least seven, at least eight, or at least nine consecutive amino acids thereof. In particular embodiments, the 7m8 insert comprises or consists of an amino acid sequence having at least 80%, at least 85%, or at least 90% homology to the amino acid sequence: LALGETTRPA (SEQ ID NO:4), or a fragment comprising at least five, at least six, at least seven, at least eight, or at least nine consecutive amino acids thereof. In one embodiment, the parental AAV capsid protein is an AAV2/7m8 capsid protein comprising or consisting of the following amino acid sequence:

(SEQ ID NO: 28)
MAADGYLPDWLEDTLSEGIRQWWKLKPGPPPPKPAERHK
DDSRGLVLPGYKYLGPFNGLDKGEPVNEADAAALEHDKA
YDRQLDSGDNPYLKYNHADAEFQERLKEDTSFGGNLGRA
VFQAKKRVLEPLGLVEEPVKTAPGKKRPVEHSPVEPDSS
SGTGKAGQQPARKRLNFGQTGDADSVPDPQPLGQPPAAP
SGLGTNTMATGSGAPMADNNEGADGVGNSSGNWHCDSTW
MGDRVITTSTRTWALPTYNNHLYKQISSQSGASNDNHYF
GYSTPWGYFDFNRFHCHFSPRDWQRLINNNWGFRPKRLN
FKLFNIQVKEVTQNDGTTTIANNLTSTVQVFTDSEYQLP
YVLGSAHQGCLPPFPADVFMVPQYGYLTLNNGSQAVGRS
SFYCLEYFPSQMLRTGNNFTFSYTFEDVPFHSSYAHSQS
LDRLMNPLIDQYLYYLSRTNTPSGTTTQSRLQFSQAGAS
DIRDQSRNWLPGPCYRQQRVSKTSADNNNSEYSWTGATK
YHLNGRDSLVNPGPAMASHKDDEEKFFPQSGVLIFGKQG
SEKTNVDIEKVMITDEEEIRTTNPVATEQYGSVSTNLQR
GNLALGETTRPARQAATADVNTQGVLPGMVWQDRDVYLQ
GPIWAKIPHTDGHFHPSPLMGGFGLKHPPPQILIKNTPV
PANPSTTFSAAKFASFITQYSTGQVSVEIEWELQKENSK
RWNPEIQYTSNYNKSVNVDFTVDTNGVYSEPRPIGTRYL
TRNL,

or a variant thereof having at least 90%, at least 95%, at least 98%, or at least 99% sequence identity to SEQ ID NO:28.

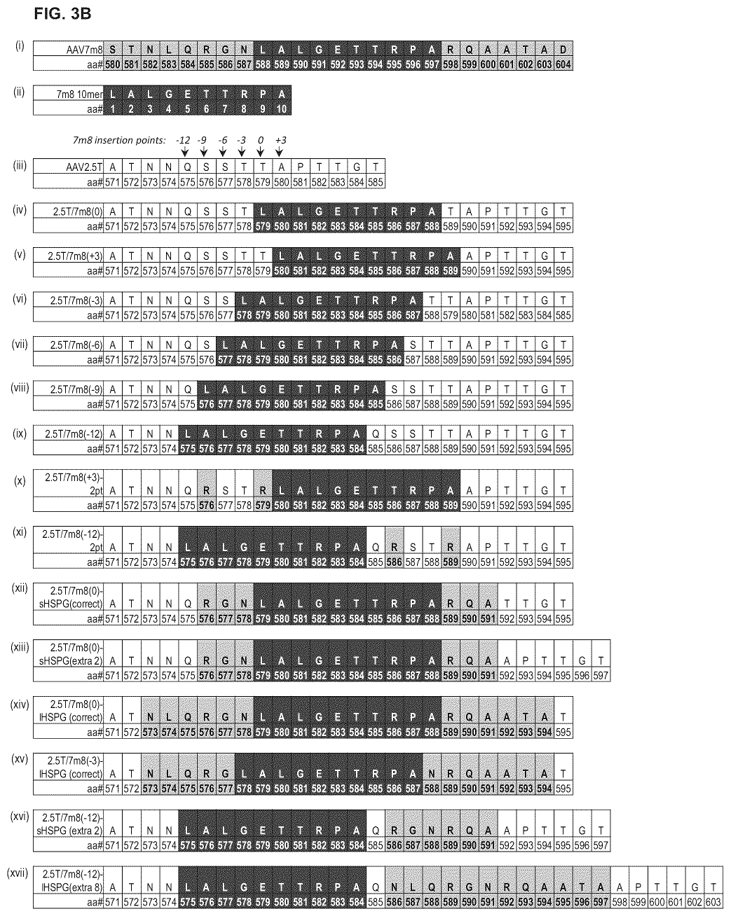

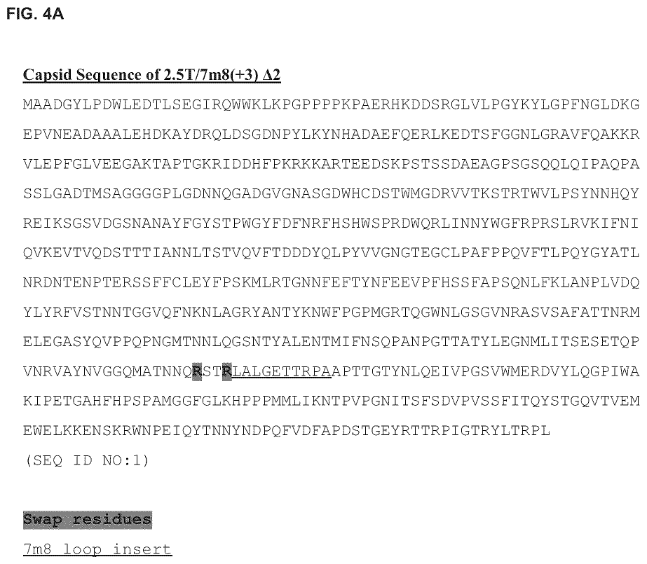

In certain embodiments, the parental capsid protein is an AAV2.5T/7m8 capsid protein, or a variant having at least 90%, at least 95%, at least 98%, at least 99% sequence identity to an AAV2.5T/7m8 capsid protein. AAV2.5T/7m8 capsid proteins correspond to AAV2.5T capsid proteins further comprising a 7m8 insert. In certain embodiment, the 7m8 amino acid insert comprises or consists of the following amino acid sequence: LGETTRP (SEQ ID NO:11). In particular embodiments, the 7m8 insert comprises or consists of the amino acid sequence: LALGETTRPA (SEQ ID NO:4), or a fragment comprising at least five, at least six, at least seven, at least eight, or at least nine consecutive amino acids thereof. In particular embodiments, the 7m8 insert comprises or consists of an amino acid sequence having at least 80%, at least 85%, or at least 90% homology to the amino acid sequence: LALGETTRPA (SEQ ID NO:4), or a fragment comprising at least five, at least six, at least seven, at least eight, or at least nine consecutive amino acids thereof. The 7m8 insert may be inserted at various sites within the AAV2.5T capsid protein. For example, it may be inserted between amino acid residues 587 and 588 of AAV2.5T as shown in FIG. 3B. Alternatively, it may be inserted either upstream or downstream, including, e.g., between any of: amino acid residues 574 and 575 (−12); amino acid residues 575 and 576 (−9); amino acid residues 576 and 577 (−6); amino acid residues 577 and 578 (−3); amino acid residues 578 and 579 (0); amino acid residues 579 and 580 (+3), as shown in FIGS. 3B and 3C. In addition, it may be inserted at other locations, including, e.g., between any of amino acid residues 571-585 of AAV2.5T.

In particular embodiments, the 7m8 insertion site is in the GH loop, or loop IV, of the AAV capsid protein, e.g., in a solvent-accessible portion of the GH loop, or loop IV, of the AAV capsid protein, such as AAV2.5T. For the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955. For example, the insertion site can be within amino acids 411-650 of an AAV capsid protein. For example, the insertion site can be within amino acids 570-611 of AAV2, within amino acids 571-612 of AAV1, within amino acids 560-601 of AAV5, within amino acids 571 to 612 of AAV6, within amino acids 572 to 613 of AAV7, within amino acids 573 to 614 of AAV8, within amino acids 571 to 612 of AAV9, or within amino acids 573 to 614 of AAV10.

In some embodiments, the insertion site is a single insertion site between two adjacent amino acids located between amino acids 570-614 of VP1 of any AAV serotype, e.g., the insertion site is between two adjacent amino acids located in amino acids 570-610, amino acids 580-600, amino acids 570-575, amino acids 575-580, amino acids 580-585, amino acids 585-590, amino acids 590-600, or amino acids 600-614, of VP1 of any AAV serotype or variant. For example, the insertion site can be between amino acids 580 and 581, amino acids 581 and 582, amino acids 583 and 584, amino acids 584 and 585, amino acids 585 and 586, amino acids 586 and 587, amino acids 587 and 588, amino acids 588 and 589, or amino acids 589 and 590. The insertion site can be between amino acids 575 and 576, amino acids 576 and 577, amino acids 577 and 578, amino acids 578 and 579, or amino acids 579 and 580. The insertion site can be between amino acids 590 and 591, amino acids 591 and 592, amino acids 592 and 593, amino acids 593 and 594, amino acids 594 and 595, amino acids 595 and 596, amino acids 596 and 597, amino acids 597 and 598, amino acids 598 and 599, or amino acids 599 and 600. For example, the insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 588 and 589 of AAV10. As another example, the insertion site can be between amino acids 450 and 460 of an AAV capsid protein. For example, the insertion site can be at (e.g., immediately N-terminal to) amino acid 453 of AAV2, at amino acid 454 of AAV1, at amino acid 454 of AAV6, at amino acid 456 of AAV7, at amino acid 456 of AAV8, at amino acid 454 of AAV9, or at amino acid 456 of AAV10. In particular embodiments of AAV2.5T/7m8 capsid proteins disclosed herein, the 7m8 insert is present between the amino acid residues in AAV2.5T corresponding to any of those described herein for other AAV, such as AAV2.

In some embodiments, a capsid protein includes an 7m8 insertion comprising an amino acid sequence having at least about 85%, at least about 90%, at least about 95%, at least about 98%, at least about 99%, or 100%, amino acid sequence identity to an amino acid sequence selected from LGETTRP (SEQ ID NO:11) and LALGETTRPA (SEQ ID NO:4).

In various embodiments, modified capsid proteins disclosed herein comprise one or more amino acid modifications as compared to a capsid protein of an AAV, e.g., AAV2.5T, AAV7m8, AAV2.5T/7m8, or AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), avian AAV, bovine AAV, canine AAV, equine AAV, primate AAV, non-primate AAV, or ovine AAV, optionally each also comprising a 7m8 insert. In certain embodiments, the one or more amino acid modification result in the modified capsid protein being capable of binding heparan or heparan sulfate proteoglycan, and certain of such variants may be referred to herein as AAV2.5T HSPG variants, AAV7m8 HSPG variants, or AAV2.5/7m8 HSPG variants, respectively.

In particular embodiments, the one or more amino acid modifications comprise introducing a heparan binding sequence of AAV2 into AAV2.5T, AAV7m8 or AAV2.5T/7m8, or any of the other AAV serotypes described herein, alone or in combination with a 7m8 insertion. In particular embodiments, the one or more amino acid modifications include one or more amino acid substitutions or insertions. In particular embodiments, variant capsid proteins disclosed herein comprise one or more amino acid substitutions corresponding to S576R or T579R when using the amino acid numbering shown in FIGS. 3A-3C for AAV2.5T.

In other embodiments, variant capsid proteins disclosed herein comprise an amino acid insertion comprising four or more, five or more, or all six consecutive amino acids within the sequence: RGNRQA (SEQ ID NO:5). In particular embodiments, the insertion comprises four or more, five or more, six or more, or all seven consecutive amino acids within the sequence: QRGNRQA (SEQ ID NO: 12). In particular embodiments, the insertion comprises four or more, five or more, six or more, seven or more, eight or more, nine or more, ten or more, or all eleven consecutive amino acids within the sequence: NLQRGNRQAATA (SEQ ID NO:7) or the sequence: NLQRGNRQAATAAP (SEQ ID NO:8). In certain embodiments, the insertion comprises or consists of an amino acid sequence having at least 80%, at least 85%, or at least 90% homology to four or more, five or more, six or more, seven or more, eight or more, nine or more, ten or more, or all eleven consecutive amino acids within the sequence: NLQRGNRQAATA (SEQ ID NO:7) or NLQRGNRQAATAAP (SEQ ID NO:8). In certain embodiments, the insertion is located upstream or downstream of the 7m8 insertion. In certain embodiments, the insertion is divided, such that one or more amino acids of the insertion are located upstream of the 7m8 insertion, and one or more amino acids of the insertion are located downstream of the 7m8 insertion (see, e.g., FIGS. 3A-3C). Accordingly, in particular embodiments, a modified AAV capsid protein, e.g., a modified AAV2.5T/7m8 capsid protein, disclosed herein comprises one or more of the following amino acid sequences, wherein the underlined sequence corresponds to a 7m8 loop insert sequence, and the non-underlined sequence corresponds to the “swap residues” amino acid sequence:

(SEQ ID NO: 13)
NLQRGNRQAATALALGETTRPA;
(SEQ ID NO: 14)
NLQRGNRQAATLALGETTRPAA;
(SEQ ID NO: 15)
NLQRGNRQAALALGETTRPATA;
(SEQ ID NO: 16)
NLQRGNRQALALGETTRPAATA;
(SEQ ID NO: 17)
NLQRGNRQLALGETTRPAAATA;
(SEQ ID NO: 18)
NLQRGNRLALGETTRPAQAATA;
(SEQ ID NO: 19)
NLQRGNLALGETTRPARQAATA;
(SEQ ID NO: 20)
NLQRGLALGETTRPANRQAATA;
(SEQ ID NO: 21)
NLQRLALGETTRPAGNRQAATA;
(SEQ ID NO: 22)
NLQLALGETTRPARGNRQAATA;
(SEQ ID NO: 23)
NLLALGETTRPAQRGNRQAATA;
(SEQ ID NO: 24)
NLALGETTRPALQRGNRQAATA;
or
(SEQ ID NO: 25)
LALGETTRPANLQRGNRQAATA.

In certain aspects, the modified AAV2.5T capsid protein comprises at least one of the one or more of the following amino acid modifications: a S576R point mutation; a T579R point mutation; a substitution of amino acid residues 576-579 or amino acid residues 576-581 with the following amino acid residues: RGNRQA (SEQ ID NO:5); a substitution of amino acid residues 576-581 with the following amino acid residues: RGNRQAAP (SEQ ID NO:6); a substitution of amino acid residues 573-581 or amino acid residues 573-584 with the following amino acid residues: NLQRGNRQAATA (SEQ ID NO:7); or a substitution of amino acid amino acid residues 573-581 or amino acid residues 573-584 with the following amino acid residues: NLQRGNRQAATAAP (SEQ ID NO:8). In particular embodiments, it comprises both a S576R point mutation and a T579R point mutation.

In particular aspects, the modified AAV2.5T/7m8 capsid protein comprises at least one of the one or more of the following amino acid modifications: a point mutation corresponding to a S576R point mutation in AAV2.5T; a point mutation corresponding to a T579R point mutation in AAV2.5T; a substitution corresponding to a substitution of amino acid residues 576-579 or amino acid residues 576-581 in AAV2.5T with the following amino acid residues: RGNRQA (SEQ ID NO:5); a substitution corresponding to a substitution of amino acid residues 576-579 or amino acid residues 576-581 in AAV2.5T with the amino acid residues: RGNRQAAP (SEQ ID NO:6) or NLQRGNRQAATA (SEQ ID NO:7); a substitution corresponding to a substitution of amino acid residues 573-579, amino acid residues 573-581, amino acid residues 573-583, or amino acid residues 573-584 in AAV2.5T with the following amino acid residues: NLQRGNRQAATA (SEQ ID NO:7); or a substitution corresponding to a substitution of amino acid residues 573-579, amino acid residues 573-581, amino acid residues 573-583, amino acid residues 573-584, or amino acid residues 576-583 in AAV2.5T with the following amino acid residues: NLQRGNRQAATAAP (SEQ ID NO:8). In particular embodiments, it comprises both a point mutation corresponding to a S576R point mutation in AAV2.5T and a point mutation corresponding to a T579R point mutation in AAV2.5T. As described herein, an AAV2.5T/7m8 capsid protein corresponds to an AAV2.5T capsid protein with an insertion of a 7m8 insertion sequence. Therefore, for convenience, modifications (e.g., amino acid substitutions or insertions) in AAV2.5T/7m8 are referred to as modifications “corresponding to” modifications in AAV2.5T, and the amino acid positions of modifications to AAV2.5T/7m8 are described by reference to the numbering of the corresponding amino acid position of AAV2.5T. The corresponding amino acid positions between AAV2.5T and AAV2.5T/7m8 can be readily determined by comparing the sequences of the capsid proteins and taking into account the presence of the 7m8 insertion in the AAV2.5T/7m8. Therefore, it is understood that the actual amino acid position number of a modification in AAV2.5T/7m8 may be different than the corresponding amino acid position number of a corresponding modification in AAV2.5T.

In certain embodiments, the modified AAV2.5T/7m8 capsid is present in a AAAV2.5T/7m8(+3), AAV2.5T/7m8(0), AAV2.5T/7m8(−3), or AAV2.5T/7m8(−12) backbone.

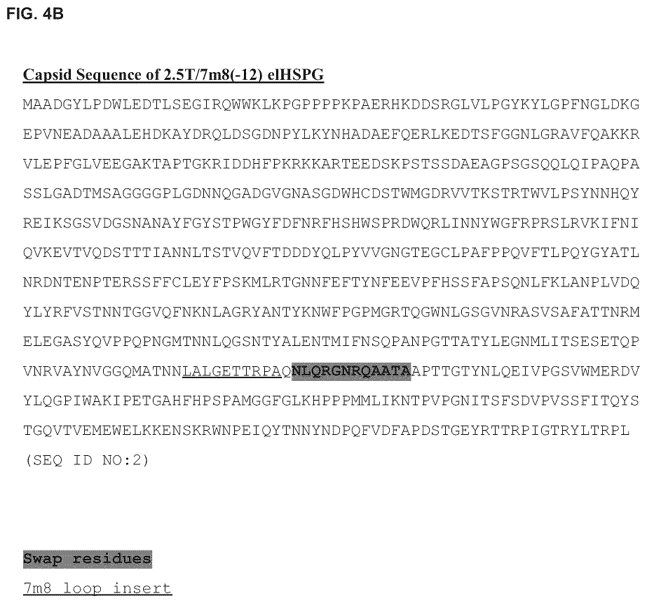

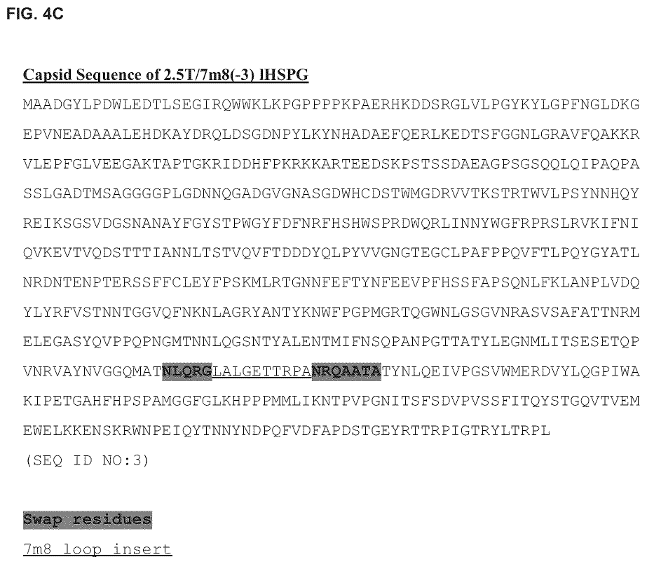

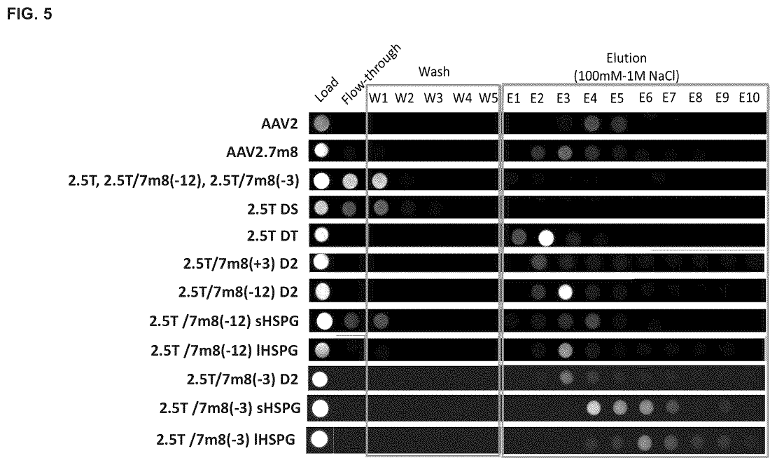

In particular embodiments, the modified AAV capsid comprises an amino acid sequence depicted in FIGS. 3A-3C or FIGS. 4A-4C. In particular embodiments, the modified AAV capsid comprises an amino acid sequence corresponding to any other AAV serotype having a modification corresponding to any of those depicted in FIGS. 3A-3C or FIGS. 4A-4C.

In certain embodiments, the variant AAV capsid protein is an AAV2.5T T579R capsid protein comprising or consisting of the following amino acid sequence:

(SEQ ID NO: 29)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNNQSSTRAPTTGTYNL
QEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNP
EIQYTNNYNDPQFVDFAPDSTGEYRTTRPIGTRYLTRPL,

or a variant thereof having at least 90%, at least 95%, at least 98%, or at least 99% sequence identity to SEQ ID NO:29.

In certain embodiments, the variant AAV capsid protein is an AAV2.5T-2pt capsid protein comprising or consisting of the following amino acid sequence:

(SEQ ID NO: 30)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNNQRSTRAPTTGTYNL
QEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNP
EIQYTNNYNDPQFVDFAPDSTGEYRTTRPIGTRYLTRPL,

or a variant thereof having at least 90%, at least 95%, at least 98%, or at least 99% sequence identity to SEQ ID NO:30.

In certain embodiments, the variant AAV capsid protein is an AAV2.5T-sHSPG(correct) capsid protein comprising or consisting of the following amino acid sequence:

(SEQ ID NO: 31)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNNQRGNRQATTGTYNL
QEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNP
EIQYTNNYNDPQFVDFAPDSTGEYRTTRPIGTRYLTRPL,

or a variant thereof having at least 90%, at least 95%, at least 98%, or at least 99% sequence identity to SEQ ID NO:31.

In certain embodiments, the variant AAV capsid protein is an AAV2.5T-IHSPG(correct) capsid protein comprising or consisting of the following amino acid sequence:

(SEQ ID NO: 32)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNLQRGNRQAATATYNL
QEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNP
EIQYTNNYNDPQFVDFAPDSTGEYRTTRPIGTRYLTRPL,

or a variant thereof having at least 90%, at least 95%, at least 98%, or at least 99% sequence identity to SEQ ID NO:32.

In certain embodiments, the variant AAV capsid protein is an AAV2.5T/7m8(0)-sHSPG(correct) capsid protein comprising or consisting of the following amino acid sequence:

(SEQ ID NO: 33)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNNQRGLALGETTRPAN
RQATTGTYNLQEIVPGSVWMERDVYLQGPIWAKIPETGAHFHPSPAMGG
FGLKHPPPMMLIKNTPVPGNITSFSDVPVSSFITQYSTGQVTVEMEWEL
KKENSKRWNPEIQYTNNYNDPQFVDFAPDSTGEYRTTRPIGTRYLTRPL,

or a variant thereof having at least 90%, at least 95%, at least 98%, or at least 99% sequence identity to SEQ ID NO:33.

In certain embodiments, the variant AAV capsid protein is an AAV2.5T/7m8(0)-IHSPG(correct) capsid protein comprising or consisting of the following amino acid sequence:

(SEQ ID NO: 34)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNLQRGNLALGETTRPA
RQAATATYNLQEIVPGSVWMERDVYLQGPIWAKIPETGAHFHPSPAMGG
FGLKHPPPMMLIKNTPVPGNITSFSDVPVSSFITQYSTGQVTVEMEWEL
KKENSKRWNPEIQYTNNYNDPQFVDFAPDSTGEYRTTRPIGTRYLTRPL,

or a variant thereof having at least 90%, at least 95%, at least 98%, or at least 99% sequence identity to SEQ ID NO:34.

In certain embodiments, the variant AAV capsid protein is an AAV2.5T/7m8(−12)-sHSPG(extra2) capsid protein comprising or consisting of the following amino acid sequence:

(SEQ ID NO: 35)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNNLALGETTRPAQRGN
RQAAPTTGTYNLQEIVPGSVWMERDVYLQGPIWAKIPETGAHFHPSPAM
GGFGLKHPPPMMLIKNTPVPGNITSFSDVPVSSFITQYSTGQVTVEMEW
ELKKENSKRWNPEIQYTNNYNDPQFVDFAPDSTGEYRTTRPIGTRYLTR
PL,

or a variant thereof having at least 90%, at least 95%, at least 98%, or at least 99% sequence identity to SEQ ID NO:35.

In certain embodiments, the variant AAV capsid protein is an AAV2.5T/7m8(+3)-2pt capsid protein comprising or consisting of the following amino acid sequence:

(SEQ ID NO: 1)
MAADGYLPDWLEDTLSEGIRQWWKLKPGPPPPKPAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPFGLVEEGAKTAPTGKRIDD
HFPKRKKARTEEDSKPSTSSDAEAGPSGSQQLQIPAQPASSLGADTMSA
GGGGPLGDNNQGADGVGNASGDWHCDSTWMGDRVVTKSTRTWVLPSYNN
HQYREIKSGSVDGSNANAYFGYSTPWGYFDFNRFHSHWSPRDWQRLINN
YWGFRPRSLRVKIFNIQVKEVTVQDSTTTIANNLTSTVQVFTDDDYQLP
YVVGNGTEGCLPAFPPQVFTLPQYGYATLNRDNTENPTERSSFFCLEYF
PSKMLRTGNNFEFTYNFEEVPFHSSFAPSQNLFKLANPLVDQYLYRFVS
TNNTGGVQFNKNLAGRYANTYKNWFPGPMGRTQGWNLGSGVNRASVSAF
ATTNRMELEGASYQVPPQPNGMTNNLQGSNTYALENTMIFNSQPANPGT
TATYLEGNMLITSESETQPVNRVAYNVGGQMATNNQRSTRLALGETTRP
AAPTTGTYNLQEIVPGSVWMERDVYLQGPIWAKIPETGAHFHPSPAMGG
FGLKHPPPMMLIKNTPVPGNITSFSDVPVSSFITQYSTGQVTVEMEWEL
KKENSKRWNPEIQYTNNYNDPQFVDFAPDSTGEYRTTRPIGTRYLTRPL,

or a variant thereof having at least 90%, at least 95%, at least 98%, or at least 99% sequence identity to SEQ ID NO:1.

In particular embodiments, the modified capsid protein is not disclosed in U.S. Pat. Nos. 9,441,244, 9,233,131, U.S. 200160017295, U.S. Pat. No. 7,220,577, WO 2015/168666, U.S. Pat. Nos. 7,867,484, 8,802,080, U.S. 20150005369, U.S. Pat. No. 7,172,893, WO 2015/134643, U.S. Pat. Nos. 6,962,815, 7,749,492, U.S. 20160040137, U.S. 20090317417, U.S. 20140336245, U.S. Pat. No. 7,629,322, WO 2016/133917, WO 2015/121501, U.S. Pat. Nos. 9,409,953, or U.S. 20150152142.

The present disclosure also includes polynucleotides that encode one or more variant capsids described herein. In particular embodiments, the polynucleotide is an expression vector, and the expression vector comprises a polynucleotide sequence encoding a variant capsid described herein operably linked to a promoter sequence, e.g., a promoter sequence that drives expression of the polynucleotide in a cell.

The present disclosure also includes cells comprising a polynucleotide or vector that encodes a variant capsid described herein. In particular embodiments, the polynucleotide is an expression vector, and the expression vector comprises a polynucleotide sequence encoding a variant capsid described herein operably linked to a promoter sequence, e.g., a promoter sequence that drives expression of the polynucleotide in the cell. In certain embodiments, the polynucleotide or vector further comprises a sequence that encodes a rep protein, e.g., an AAV2 rep protein. In certain embodiments, the cell is a helper cell or host cell, such as, e.g., an HEK293 cell that may be used to produce virions comprising the variant capsid protein. In preparing the subject rAAV compositions, any host cells for producing rAAV virions may be employed, including. for example, mammalian cells (e.g. 293 cells), insect cells (e.g. SF9) cells), microorganisms and yeast. Host cells can also be packaging cells in which the AAV rep and cap genes are stably maintained in the host cell or producer cells in which the AAV vector genome is stably maintained and packaged. Exemplary packaging and producer cells are derived from SF-9, 293, A549 or HeLa cells. AAV vectors are purified and formulated using standard techniques known in the art.

### Recombinant Virions and Viral Vectors

The present invention includes recombinant viruses or virions, e.g., gene delivery vectors or gene therapy vectors, that comprise a variant capsid protein of the present disclosure.

In certain embodiments, the virus or virion is a viral vector derived from a virus, e.g., an adenovirus, an adeno-associated virus (AAV), a lentivirus, a herpes virus, an alpha virus or a retrovirus, e.g., Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) or lentivirus. While embodiments encompassing the use of adeno-associated virus are described in greater detail below, it is expected that the ordinarily skilled artisan will appreciate that similar knowledge and skill in the art can be brought to bear on non-AAV gene therapy vectors as well. See, for example, the discussion of retroviral vectors in, e.g., U.S. Pat. Nos. 7,585,676 and 8,900,858, and the discussion of adenoviral vectors in, e.g. U.S. Pat. No. 7,858,367, the full disclosures of which are incorporated herein by reference. In certain embodiments, the recombinant virus or virion is infectious. In certain embodiments, the recombinant virion or virus is replication-competent. In certain embodiments, the recombinant virus or virion is replication-incompetent. In particular embodiments, the virion is an AAV2.5T, an AAV7m8 or an AAV2.5T/7m8 comprising a modified capsid protein described herein.

In some embodiments, the recombinant virion or virus , e.g., an AAV, further comprises a polynucleotide cassette comprising a sequence that encodes a gene product, e.g., a therapeutic gene product. In certain embodiments, the polynucleotide cassette is flanked on the 5′ and 3′ ends by functional AAV inverted terminal repeat (ITR) sequences. By “functional AAV ITR sequences” is meant that the ITR sequences function as intended for the rescue, replication and packaging of the AAV virion. Hence, AAV ITRs for use in the gene delivery vectors of the invention need not have a wild-type nucleotide sequence, and may be altered by the insertion, deletion or substitution of nucleotides or the AAV ITRs may be derived from any of several AAV serotypes, e.g. AAV1, AAV2, AAV3, AAV4, AAV5, AAV6, AAV7, AAV8, AAV9), AAV10. Preferred AAV vectors have the wild type REP and CAP genes deleted in whole or part, but retain functional flanking ITR sequences.

Recombinant viral vectors (e.g., rAAV virions) comprising variant capsid proteins described herein, and optionally encapsulating polynucleotide cassettes of the present disclosure, may be produced using standard methodology. For example, in the case of rAAV virions, an AAV expression vector comprising a polynucleotide cassette may be introduced into a producer cell, followed by introduction of an AAV helper construct comprising a polynucleotide sequence encoding a variant capsid protein disclosed herein, and where the helper construct includes AAV coding regions capable of being expressed in the producer cell and which complement AAV helper functions absent in the AAV vector. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions comprising variant capsid proteins described herein are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety.

As disclosed in the accompanying Example, variant capsid proteins described herein confer enhanced or altered cellular tropism to virions comprising the variant capsid proteins. Accordingly, the variant capsids may be used to enhance or alter the tropism of a virus or virion in order to enhance its tropism for a desired cell type.

In particular embodiments, the virions or viral vectors comprising a variant capsid protein described herein bind to heparan or heparan sulfate proteoglycans (HSPGs), e.g., with an affinity at least 2-fold, at least 3-fold, at least 4-fold, at least 5-fold, at least 10-fold, at least 20-fold, at least 50-fold, or at least 100-fold as compared to a corresponding virion or viral vector that does not include a variant capsid protein disclosed herein.

In particular embodiments, the virions or viral vectors comprising a variant capsid protein described herein bind to ILM, e.g., with an affinity at least 2-fold, at least 3-fold, at least 4-fold, at least 5-fold, at least 10-fold, at least 20-fold, at least 50-fold, or at least 100-fold as compared to a corresponding virion or viral vector that does not include a variant capsid protein disclosed herein.

In particular embodiments, the virions or viral vectors comprising a variant capsid protein described herein are capable of delivering a gene product to the retina when delivered via intravitreal injection, e.g., wherein they result in the expression of at least 2-fold, at least 3-fold, at least 4-fold, at least 5-fold, at least 10-fold, at least 20-fold, at least 50-fold, or at least 100-fold gene product as compared to a corresponding virion or viral vector that does not include a variant capsid protein disclosed herein.

In certain embodiments, the virions or viral vectors bind heparan or heparan sulfate with a binding affinity such that they are eluted from a heparan column at a salt concentration of about 0.2 M to about 0.4 M, e.g., as described in the accompanying Examples.

### Pharmaceutical Compositions

Pharmaceutical compositions comprising a virion or viral vector comprising a variant capsid protein described herein and one or more pharmaceutically acceptable diluent. carrier, or excipient are also disclosed. The subject virions or vector can be combined with pharmaceutically-acceptable carriers, diluents and reagents useful in preparing a formulation that is generally safe, non-toxic, and desirable, and includes excipients that are acceptable for primate use. Such excipients can be solid, liquid, semisolid, or, in the case of an aerosol composition, gaseous. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite: chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile. For instances in which ocular cells are to be contacted in vivo, the subject polynucleotide cassettes or gene delivery vectors comprising the subject polynucleotide cassette can be treated as appropriate for delivery to the eye.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid to the extent that easy syringability exists. In certain embodiments, it is stable under the conditions of manufacture and storage and is preserved against the contaminating action of microorganisms such as bacteria and fungi. The carrier can be, e.g., a solvent or dispersion medium containing. for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the vector or virion in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the virion or vector against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It may be advantageous to formulate oral, ocular or parenteral compositions in dosage unit form for ease of administration and uniformity of dosage. Dosage unit form as used herein refers to physically discrete units suited as unitary dosages for the subject to be treated: each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Any concentration of viral particles suitable to effectively transducer mammalian cells can be prepared. For example, the viral particles may be formulated at a concentration of 10⁸ vector genomes per ml or more, for example, 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 1×10¹⁴ vector genomes per mL, 5×10¹⁴ vector genomes per mL or more, but typically not more than 1×10¹⁵ vector genomes per mL.

The subject viral vector may be formulated into any suitable unit dosage, including. without limitation, 1×10⁸ vector genomes or more, for example, 1×10⁹, 1×10¹⁰, 1×10¹¹, 1×10¹², or 1×10¹³ vector genomes or more, in certain instances, 1×10¹⁴ vector genomes, but usually no more than 4×10¹⁵ vector genomes. In some cases, the unit dosage is at most about 5×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×108 vector genomes. In some cases, the unit dosage is 1×10¹⁰ to 1×10¹¹ vector genomes. In some cases, the unit dosage is 1×10¹⁰ to 3×10¹² vector genomes. In some cases, the unit dosage is 1×10⁹ to 3×10¹³ vector genomes. In some cases, the unit dosage is 1×10⁸ to 3×10¹⁴ vector genomes.

In some cases, the unit dosage of a pharmaceutical composition may be measured using multiplicity of infection (MOI). By MOI it is meant the ratio, or multiple, of vector or viral genomes to the cells to which the nucleic acid may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some aspects, the amount of pharmaceutical composition comprises about 1×10⁸ to about 1×10¹⁵ recombinant viruses, about 1×10⁹ to about 1×10¹⁴ recombinant viruses, about 1×10¹⁰ to about 1×10¹³ recombinant viruses, or about 1×10¹¹ to about 3×10¹² recombinant viruses.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active metabolite or residue thereof.

The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N-N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

The subject polynucleotide cassette or gene delivery vector, e.g., recombinant virus (virions), can be incorporated into pharmaceutical compositions for administration to mammalian patients, particularly primates and more particularly humans. The subject polynucleotide cassette or gene delivery vector, e.g. virions can be formulated in nontoxic, inert, pharmaceutically acceptable aqueous carriers, preferably at a pH ranging from 3 to 8, more preferably ranging from 6 to 8. Such sterile compositions will comprise the vector or virion containing the nucleic acid encoding the therapeutic molecule dissolved in an aqueous buffer having an acceptable pH upon reconstitution.

In some embodiments, the pharmaceutical composition provided herein comprise a therapeutically effective amount of a vector or virion in admixture with a pharmaceutically acceptable carrier and/or excipient. for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer in which the pharmaceutical composition comprising the tumor suppressor gene contained in the adenoviral vector delivery system, may be in the range of 6.5 to 7.75, preferably 7 to 7.5, and most preferably 7.2 to 7.4.

### Method of Enhancing or Altering Viral Tropism

As disclosed in the accompanying Examples, variant capsid proteins described herein confer enhanced or altered cellular tropism to virions comprising the variant capsid proteins. For example, certain variant capsid proteins described herein are associated with increased infectivity of the retina or increased expression levels of gene product in the retina, increased binding to the ILM, increased infectivity of the retina following intravitreal injection or increased expression of gene product in the retina following intravitreal injection, and/or increased infectivity of or increased expression of gene products in particular ocular cells, such as, e.g., retinal cells, RGC or Müller cells.

Examples of retinal cell components that may be transfected by vectors of the instant invention can include, but are not limited to, astrocytes, Müller Cells, RGC, RGC axons, photoreceptors, bipolar cells, amacrine cells, horizontal cells and combinations thereof. In some instances, cell markers are used to measure transduction of a transgene into a retinal cell component. Cell markers can include, but are not limited to, GFAP, Vimetin, Fox, β-tubulin, rhodopsin, PKCa, parvalbumin, calbinding and combinations thereof. Table 1 below depicts each cell marker with the corresponding retinal cell component.

TABLE 1
Summary of cell markers and cell types

| Marker | Cell Type |
|---|---|
| GFAP | Astrocytes |
| Vimentin | Müller Cells |
| Fox | RGC |
| B-tubulin | RGC axons |
| Rhodopsin (Rho) | Photoreceptors |
| PKCa | Bipolar cells |
| Parvalbumin | Amacrine cells |
| Calbindin | Horizontal cells |

In some instances, the ability of an rAAV virion to transduce a transgene can be measured by the expression of a reporter protein. In some embodiments, the reporter protein can include GFP, YFP, red cherry, B-galactosidase, or other reporter proteins known in the art.

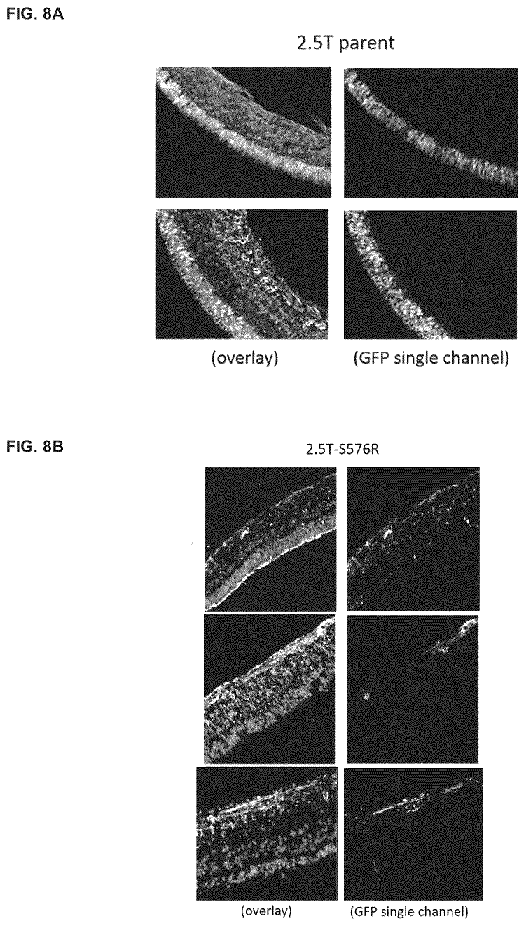

In some instances, the expression of the reporter protein over time can be determined through a capture of an image of an eye at various time points. As an exemplary example, the expression of GFP can be correlated to an increase in fluorescence. FIG. 7 (lower panels) depicts expression of GFP in the fundus of gerbils at eight weeks after intravitreal injection of various rAAV virions. The amount of fluorescence at a given time point can be used to quantitate expression of a transgene.

In some embodiments, immunofluorescence can be used to determine localization of a transduced gene in a retinal cell compartment described herein. The retinal cell compartment can be labeled by use of an antibody specific for a cell marker disclosed herein. In some instances, the antibody is coupled to a fluorophore. In some instances, a secondary antibody can be used to detect binding of the first antibody. The transduced retina can be cyrosectioned prior to incubation with an antibody against the cell marker of interest, thereby allowing imaging of the bound antibody. The cryosection can then be labeled using antibodies to detect the transduction of a transgene, the presence of a cell maker of interest, and regions of overlap between the two.

The variant capsids disclosed herein may be used to enhance or alter the tropism of a virus or virion in order to enhance its tropism for a desired cell type.

In particular embodiments, the virions or viral vectors comprising a variant capsid protein described herein are used to deliver a gene product to the retina.

In particular embodiments, the virions or viral vectors comprising a variant capsid protein described herein are used to deliver a gene product across the ILM.

In particular embodiments, the virions or viral vectors comprising a variant capsid protein described herein are used to deliver a gene product to the retina by intravitreal injection.

In certain embodiment, the disclosure provides a method of altering the tropism of a virus, e.g., an AAV2.5T or AAV2.5T/7m8 virus, comprising introducing one or more amino acid modifications that confers heparan sulfate binding to a capsid protein of the virus. In related embodiments, the method comprises a method of altering the tropism of a virus, comprising incorporating a variant capsid protein disclosed herein into the virus. In particular embodiments, the alteration comprises one or more amino acid modification introduces one or more amino acids of AAV2 into the capsid protein, including, e.g., any of the modifications described herein. In certain embodiments, the one of the one or more amino acid modifications comprises: (a) a point mutation corresponding to a S576R point mutation in AAV2.5T; (b) a point mutation corresponding to a T579R point mutation in AAV2.5T; (c) a substitution corresponding to a substitution of amino acid residues 576-581 in AAV2.5T with the following amino acid residues: RGNRQA (SEQ ID NO:5); or (d) a substitution corresponding to a substitution of amino acid residues 573-584 in AAV2.5T with the following amino acid residues: NLQRGNRQAATA (SEQ ID NO:7).

In certain embodiments, the method comprises altering the tropism of a virion or viral vector to increase infectivity of RGC as compared to AAV2.5T or AAV2.5T/7m8, wherein the amino acid modification comprises: (a) a S576R point mutation; or (b) a T579R point mutation, e.g., an amino acid mutation corresponding to S576R or T579R in AAV2.5T. Accordingly, the present disclosure comprises a method for selectively delivering a gene product, e.g., a polypeptide, to RGC of a subject, comprising administering to the subject an AAV2.5T virion comprising a capsid protein comprising a S576R point mutation or a T579R point mutation. In certain embodiments, the viral vector is AAV2.5T-S576R or AAV2.5T-T579R.

In certain embodiments, the method comprises altering the tropism of a virion or viral vector to increase infectivity of Müller cells as compared to AAV2.5T or AAV2.5/7m8, wherein the amino acid modification comprises: (a) a S576R point mutation; and (b) a T579R point mutation, e.g., amino acid mutation corresponding to S576R and T579R in AAV2.5T. Accordingly, the present disclosure comprises a method for selectively delivering a gene product, e.g., polypeptide, to Müller cells of a subject, comprising administering to the subject an AAV2.5T/7m8 virus comprising a polynucleotide that encodes the polypeptide. where the virus comprises a capsid protein comprising: (a) a S576R point mutation; and (b) a T579R point mutation. In certain embodiments, the viral vector is AAV2.5T/7m8(+3)Δ2, as described herein.

In certain embodiments, the method comprises altering the tropism of a virion or viral vector to increase infectivity of retinal cells as compared to AAV2.5T or AAV2.5T/7m8 (or variants thereof), wherein the amino acid modification comprises a substitution of amino acid residues corresponding to 576-579 in AAV2.5T with the following amino acid residues: NLQRGNRQAATA (SEQ ID NO:7) or substitution of amino acid residues corresponding to 573-579 or 573-584 with the following amino acid residues: TNQNLQRGNRQAATA (SEQ ID NO:9. Accordingly, in certain embodiments, the present disclosure provides a method for increasing delivery of a gene product, e.g., polypeptide, to retinal cells of a subject, comprising administering to the subject an AAV2.5T/7m8 virus comprising a polynucleotide that encodes the polypeptide, where the virus comprises a capsid protein comprising a substitution of amino acid residues corresponding to 576-579 in AAV2.5T with the following amino acid residues: NLQRGNRQAATA (SEQ ID NO:7) or substitution of amino acid residues corresponding to 573-579 or 573-584 with the following amino acid residues: TNQNLQRGNRQAATA (SEQ ID NO:9). In certain embodiments, the viral vector is AAV2.5T/7m8(−12)-elHSPG (also referred to as AAV2.5T/7m8−12-lHSPG(extra8)), as described herein.

### Methods of Expressing Gene Products and Treating Diseases And Disorders

Virions and viral vectors described herein, comprising a variant capsid protein described herein, may be used in delivering a transgene to a cell, e.g., cells of an animal. For example, they may be used in research, e.g., to determine the effect that the gene has on cell viability and/or function. As another example, they may be used in medicine, e.g. to treat a disorder, for example, by delivering a therapeutic gene product to a cell or tissue. Thus, in some aspects of the invention, methods are provided for the expression of a gene in cells, the method comprising contacting cells with a composition of the present disclosure. In some embodiments, contacting occurs in vitro. In some embodiments, contacting occurs in vivo, i.e., the subject composition is administered to a subject. In particular embodiments, a viral vector is administered parenterally, e.g., intravenously, orally, or by injection. In certain embodiments, it is administered to the eye by injection, e.g., administered to the retina, sub-retina or vitreous. In certain embodiments, it is administered by retinal injection, sub-retinal injection, or intravitreal injection.

For instances in particular embodiments in which mammalian cells are to be contacted in vitro with a subject virion or vector comprising a variant capsid protein disclosed herein, the cells may be from any mammalian species, e.g. rodent (e.g. mice, rats, gerbils, squirrels), rabbit, feline, canine, goat, ovine, pig, equine, bovine, primate, human. Cells may be from established cell lines, e.g. WERI cells, 661W cells, or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present disclosure are maintained for fewer than 10 passages in vitro.

If the cells are primary cells, they may be harvested from a mammal by any convenient method, e.g. whole explant, biopsy, etc. An appropriate solution may be used for dispersion or suspension of the harvested cells. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

In certain embodiments, to promote expression of the transgene, the subject virion or gene delivery vector comprising a variant capsid protein is contacted with the cells for about 30 minutes to 24 hours or more, e.g., 1 hour, 1.5 hours, 2 hours, 2.5 hours, 3 hours, 3.5 hours 4 hours. 5 hours, 6 hours, 7 hours, 8 hours, 12 hours, 16 hours, 18 hours, 20 hours, 24 hours, etc. The subject virion or gene delivery vector comprising a variant capsid protein may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further. Contacting the cells may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

In certain embodiments, an effective amount of a virion or gene delivery vector comprising a variant capsid protein is provided to produce the expression of the transgene in cells. In particular embodiments, the effective amount may be readily determined empirically, e.g., by detecting the presence or levels of transgene gene product, by detecting an effect on the viability or function of the cells, etc. In certain embodiments, an effect amount of subject virion or gene delivery vector comprising a variant capsid protein will promote equal or greater expression of the transgene in cells than the same amount of a reference virion or viral vector known in the art, e.g., AAV2.5T or AAV7m8. In certain embodiments, expression is enhanced 2-fold or more relative to the expression from a reference, or control virion or viral vector, for example 3-fold, 4-fold, or 5-fold or more, in some instances 10-fold, 20-fold or 50-fold or more, e.g. 100-fold. In certain embodiments, the enhanced expression occurs is a particular cell type, e.g., any of the ocular cells described herein.

For instances in which cells are contacted in vivo with a subject virion or gene delivery vector comprising a variant capsid protein described herein, the subject may be any mammal, e.g. rodent (e.g. mice, rats, gerbils), rabbit, feline, canine, goat, ovine, pig, equine, bovine, or primate (e.g. human, or non-human primate). The methods and compositions of the present disclosure find use in the treatment of any condition that can be addressed, at least in part, by gene therapy of cells. Thus, the compositions and methods of the present disclosure find use in the treatment of individuals in need of a cell therapy. Cells include but are not limited to blood, eye, liver, kidney, heart, muscle, stomach, intestine, pancreas, and skin.

In certain embodiments, the disclosure provides a method of providing a gene product to a retina of a subject, comprising administering to the subject by intravitreal injection a pharmaceutical composition comprising a recombinant virion or vector described herein, wherein the recombinant virus comprises a variant capsid provide disclosed herein and a polynucleotide sequence that encodes the gene product.

In particular embodiments, the subject has been diagnosed with or is suspected of having one or more diseases or disorders selected from the group consisting of: age-related macular degeneration (AMD), wet-AMD, dry-AMD, retinal neovascularization, choroidal neovascularization, diabetic retinopathy, proliferative diabetic retinopathy, retinal vein occlusion, central retinal vein occlusion, branched retinal vein occlusion, diabetic macular edema, diabetic retinal ischemia, ischemic retinopathy, and diabetic retinal edema.

In certain embodiments, the gene product inhibits neovascularization, e.g., choroidal neovascularization (CNV), in the retina of the subject. It has been found that many cellular factors play important roles in regulation in CNV generation, among which may include but are not limited to vascular endothelial growth factor (VEGF), VEGF receptor (VEGFR), platelet-derived growth factor (PDGF), hypoxia inducible factor (HIF), angiopoietin (Ang) and other cytokines, mitogen-activated protein kinases (MAPK). In particular embodiments, the gene product inhibits one or more of these cellular factors.

In particular embodiments, the gene product is an anti-VEGF protein, such as, but not limited to the VEGF-binding proteins or functional fragments thereof disclosed in U.S. Pat. Nos. 5,712,380, 5,861,484 and 7,071,159 and VEGF-binding fusion proteins disclosed in U.S. Pat. No. 7,635,474. An anti-VEGF protein may also include the soluble Fms-related tyrosine kinase-1 (sFLT-1) protein as described in U.S. Patent Application Publication No. 2013/0323302.

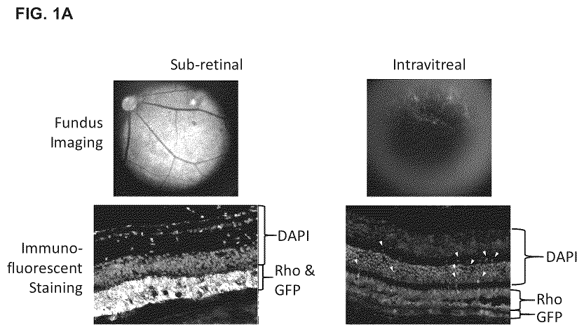

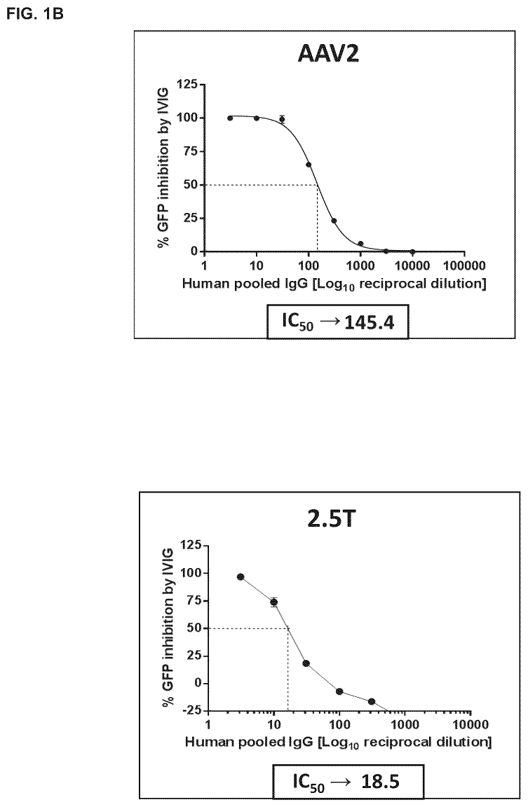

The recombinant virions and viral vectors of the present disclosure may comprise a sequence encoding an anti-VEGF protein, including but not limited to anti-VEGF antibodies or fragments thereof, such as aflibercept, ranibizumab, and bevacizumab, and the naturally occurring protein sFlt-1, as described in U.S. Pat. No. 5,861,484 and the sequence disclosed by SEQ ID NO: 109 in US2013/0323302. An sFlt-1 amino acid sequence is disclosed herein as SEQ ID NO:42. It also includes, but is not limited to functional fragments thereof, including sequences of sFlt-1 domain 2 or those set forth in SEQ ID NO: 121 of U.S. Patent Application Publication No. 2013/0323302, as well as related constructs, such as the VEGF-binding fusion proteins disclosed in U.S. Pat. No. 7,635,474. An s-Flt-1 functional fragment is disclosed herein as SEQ ID NO:43. An anti-VEGF protein may also include any of the sFLT-1 proteins, variants or fragments thereof described in U.S. Patent Application Publication No. 2013/0323302. In some embodiments, the anti-VEGF protein is ranibizumab (commercially available under the trademark Lucentis® (Genentech, San Francisco, CA), see FIG. 1 of U.S. Pat. No. 7,060,269 for the heavy chain and light chain variable region sequences of ranibizumab); bevacizumab (commercially available under the trademark Avastin® (Genentech, San Francisco, CA), see FIG. 1 of U.S. Pat. No. 6,054,297 for the heavy chain and light chain variable region sequences of bevacizumab); or aflibercept (commercially available under the trademark Eylea® (Regeneron, Tarrytown, NY). In certain embodiments, bevacizumab includes the following heavy and light chain variable domain sequences, respectively:

(SEQ ID NO: 36)
EVQLVESGGGLVQPGGSLRLSCAASGYTFTNYGMNWVRQAPGKGLEWVG
WINTYTGEPTYAADFKRRFTFSLDTSKSTAYLQMNSLRAEDTAVYYCAK
YPHYYGSSHWYFDVWGQGTL;
and
(SEQ ID NO: 37)
DIQMTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTV.

In certain embodiments, ranibizumab includes the following heavy and light chain variable domain sequences, respectively:

(SEQ ID NO: 38)
EVQLVESGGGLVQPGGSLRLSCAASGYDFTHYGMNWVRQAPGKGLEWVG
WINTYTGEPTYAADFKRRFTFSLDTSKSTAYLQMNSLRAEDTAVYYCAK
YPYYYGTSHWYFDVWGQGTL;
and
(SEQ ID NO: 39)
DIQLTQSPSSLSASVGDRVTITCSASQDISNYLNWYQQKPGKAPKVLIY
FTSSLHSGVPSRFSGSGSGTDFTLTISSLQPEDFATYYCQQYSTVPWTF
GQGTKVEIKRTV.

In certain embodiments, aflibercept includes the following amino acid sequence:

(SEQ ID NO: 40)
MVSYWDTGVLLCALLSCLLLTGSSSGSDTGRPFVEMYSEIPEIIHMTEG
RELVIPCRVTSPNITVTLKKFPLDTLIPDGKRIIWDSRKGFIISNATYK
EIGLLTCEATVNGHLYKTNYLTHRQTNTIIDVVLSPSHGIELSVGEKLV
LNCTARTELNVGIDFNWEYPSSKHQHKKLVNRDLKTQSGSEMKKFLSTL
TIDGVTRSDQGLYTCAASSGLMTKKNSTFVRVHEKDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHE
ALHNHYTQKSLSLSPGK.

These sequences can be expressed from DNA encoding such sequences using the genetic code, a standard technique that is understood by those skilled in the art. As can be appreciated by those with skill in the art, due to the degeneracy of the genetic code, anti-VEGF protein sequences can be readily expressed from a number of different DNA sequences.

“sFlt-1 protein” herein refers to a polypeptide sequence, or functional fragment thereof, with at least 90%, or more, homology to the naturally occurring human sFLT-1 sequence, such that the sFlt-1 protein or polypeptide binds to VEGF and/or the VEGF receptor. Homology refers to the % conservation of residues of an alignment between two sequences (e.g. as Naturally occurring human sFLT-1 protein may include any suitable variants of sFLT-1, including, but not limited to functional fragments, sequences comprising insertions, deletions, substitutions, pseudofragments, pseudogenes, splice variants or artificially optimized sequences. In some cases, “sFLT-1 protein” may be at least about 90%, 91%, 92%, 93%, 94%, 95%, 96%, 97%, 98%, 99%, 99.9%, 99.99% or 100% homologous to the naturally occurring human sFLT-1 protein sequence. In some cases, “sFLT-1 protein” may be at most about 90%, 91%, 92%, 93%, 94%, 95%, 96%, 97%, 98%, 99%, 99.9%, 99.99% or 100% homologous to the naturally occurring human sFLT-1 protein sequence. In some cases, “sFLT-1 protein” may be at least about 90%, 91%, 92%, 93%, 94%, 95%, 96%, 97%, 98%, 99%, 99.9%, 99.99% or 100% spatially homologous to the naturally occurring human sFLT-1 protein conformation. In some cases, “sFLT-1 protein” may be at most about 90%, 91%, 92%, 93%, 94%, 95%, 96%, 97%, 98%, 99%, 99.9%, 99.99% or 100% spatially homologous to the naturally occurring human sFLT-1 protein conformation.

Further, the soluble truncated form of the VEGF receptor FLT-1, sFLT-1, is the only known endogenous specific inhibitor of VEGF. In nature, it is generated by alternative mRNA splicing and lacks the membrane-proximal immunoglobulin-like domain, the transmembrane spanning region and the intracellular tyrosine-kinase domain. Structurally, FLT-1 and sFLT-1 protein may both comprise multiple functional domains. In some variants, FLT and sFLT proteins commonly share 6 interlinked domain: 3 domains involved in dimerization of the protein and 3 domains involved in the binding of a ligand, such as VEGF.

sFLT-1 is a soluble truncated form of the FLT-1 and it is expressed endogenously. As described herein, “soluble” FLT-1, or sFLT-1 refers to FLT-1 that is not restricted to the cellular membrane. Unbound sFLT-1 may diffuse freely in extracellular space or solution.

sFLT-1 is the only known endogenous specific inhibitor of VEGF. This interaction is specific and can be competed away with 100-fold excess unlabeled VEGF. In some cases, the angiostatic activity of sFLT-1 may result from inhibition of VEGF by two mechanisms: i) sequestration of VEGF, to which it binds with high affinity, and ii) formation of inactive heterodimers with membrane-spanning isoforms of the VEGF receptors FLTt-1 and FLK-1/KDR. As known in the art, in vitro binding assays have indicated that sFLT-1 binds VEGF with high affinity and may also inhibit VEGF driven proliferation of human umbilical vein endothelial cells. In animal models for cancer, sFLT-1 inhibits tumor growth. In some cases, sFLT-1 may function in a substoichiometric or dominant negative manner, as excess VEGF in the extracellular space may be prevented from binding and subsequently activating the VEGF receptor. These properties of sFLT-I have been described in Kendall and Thomas, 1993; Proc Natl Acad Sci. 90: 10705-10709, which is incorporated herein by reference in its entirety. As is known in the art, functional fragments of sFLT-1 can be used in place of the full-length protein. More specifically, the VEGF binding domain (domain 2), or alternatively domain 2 of sFLT-1 plus domain 3 from sFLTI, KDR, or another family member, can be used to bind and inactivate VEGF. Such functional fragments are described in Wiesmann et al., 1997; Cell, 91: 695-704, which is incorporated herein by reference in its entirety. The terms “sFLT-1” and “a functional fragment of sFLT-1” are equivalent and used here interchangeably.

In certain embodiments, virions and viral vectors comprising a variant capsid protein that confers altered tropism to the virion or viral vector is used to treat a disease or disorder of the cells for which tropism or the virion or viral vector is increased.

In certain embodiments, a virion or viral vector comprising a variant capsid protein comprising a S576R point mutation or a T579R point mutation is administered (e.g., via intravitreal injection) to a subject to treat a disease or disorder associated with retinal ganglial cells (RGC), or to deliver a therapeutic gene product to RGC. In particular embodiments, the virion or viral vector is an AAV2.5T or AAV2.5T/7m8 virion comprising a capsid protein comprising a S576R point mutation or a T579R point mutation. In certain embodiments, the virion or viral vector comprises a S576R point mutation or a T579R point mutation, but not both. In certain embodiments, the disease or disorder is glaucoma or a neurodegenerative disease.

In certain embodiments, a virion or viral vector comprising a variant capsid protein comprising a S576R point mutation and a T579R point mutation is administered (e.g., via intravitreal injection) to a subject to treat a disease or disorder associated with Müller cells, or to deliver a therapeutic gene product to Müller cells. In particular embodiments, the virion or viral vector is an AAV2.5T or AAV2.5T/7m8 virion comprising a capsid protein comprising a S576R point mutation and a T579R point mutation. In certain embodiments, the disease or disorder is a retinal disease.

In certain embodiments, a virion or viral vector comprising a substitution of amino acid residues corresponding to 573-584 in AAV2.5T/7m8 with the following amino acid residues: NLQRGNRQAATA (SEQ ID NO:7) is administered (e.g., via intravitreal injection) to a subject to treat a disease or disorder associated with retinal cells. In particular embodiments, the virion or viral vector is an AAV2.5T or AAV2.5T/7m8 virion comprising a capsid protein comprising a substitution of amino acid residues corresponding to 573-584 in AAV2.5T/7m8 with the following amino acid residues: NLQRGNRQAATA (SEQ ID NO:7). In particular embodiments, the disease or disorder is a retinal disease.

In some embodiments, the subject method results in a therapeutic benefit. e.g. preventing the development of a disorder, halting the progression of a disorder, reversing the progression of a disorder, etc. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

In some instances, the expression of the transgene, e.g. as detected by measuring levels of gene product, by measuring therapeutic efficacy, etc., may be observed two months or less after administration, e.g. 4, 3 or 2 weeks or less after administration, for example, 1 week after administration of the subject composition. Expression of the transgene is also expected to persist over time. Accordingly, in some instances, the expression of the transgene, e.g., as detected by measuring levels of gene product, by measuring therapeutic efficacy, etc., may be observed 2 months or more after administration of the subject composition, e.g., 4, 6, 8, or 10 months or more, in some instances 1 year or more, for example 2, 3, 4, or 5 years, in certain instances, more than 5 years.

In particular embodiments, a subject is administered to one eye or to each of both eyes about 1×10⁸ vector genomes or more, in some cases 1×10⁹, 1×10¹⁰, 1×10¹¹, 1×10¹², or 1×10¹³ vector genomes or more, in certain instances, 1×10¹⁴ vector genomes or more. In some cases, the amount of vector genomes that is delivered is at most about 1×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes, and sometimes no less than 1×10⁸ vector genomes. In some cases, the amount of vector genomes that is delivered is 1×10¹⁰ to 1×10¹¹ vector genomes. In some cases, the amount of vector genomes that is delivered is 1×10¹⁰ to 3×10¹² vector genomes. In some cases, the amount of vector genomes that is delivered is 1×10⁹ to 3×10¹³ vector genomes. In some cases, the amount of vector genomes that is delivered is 1×10⁸ to 3×10¹⁴ vector genomes.

In some cases, the amount of pharmaceutical composition to be administered may be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some aspects, the amount of pharmaceutical composition comprises about 1×10⁸ to about 1×10¹⁵ particles of recombinant virions or viruses, about 1×10⁹ to about 1×10¹⁴ particles of recombinant virions or viruses, about 1×10¹⁰ to about 1×10¹³ particles of recombinant virions or viruses, or about 1×10¹¹ to about 3×10¹² particles of recombinant virions or viruses.

In some aspects, no virion or vector is detected in the human subject's tear, blood, saliva or urine samples 7, 14, 21 or 30 days after administering said pharmaceutical composition. In some aspects, the presence of the viral vector is detected by qPCR or ELBA as known in the art.

In some aspects, a subject's best corrected visual acuity (BCVA) improves by 1, 2 3, 4, 5 or more lines following a method of treatment described herein.

In some aspects, a reduction in neovascularization as assessed by Fluorscein Angiography (FA) follows the administering step.

In some cases, retinal thickness may be measured to examine the effects of treatment. In some cases, the central retinal thickness of the human subject does not increase by more than 50 microns, 100 microns, or 250 microns within 12 months following treatment with the pharmaceutical composition of the disclosure. In some cases, the central retinal thickness of the human subject decreases by at least 50 microns, 100 microns, 200 microns, 250 microns, 300 microns, 400 microns, 500 microns, 600 microns within 3 months, 6 months or 9 months 12 months following treatment with the pharmaceutical composition of the disclosure. The decrease in the central retinal thickness of the human subject may be measured comparing the central retinal thickness at point in time to a baseline measurement taken at or within 1, 3, 7 or 10 days of the administration of the pharmaceutical composition of the disclosure.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

### Construction of AAV2.5T Swap Mutants

AAV2.5T is a hybrid capsid containing regions from AAV2 and AAV5. AAV2.5T is capable of transducing the retina when delivered subretinally, but not when injected intravitreally, see FIG. 1A. AAV2.5T transduction may be blocked by the ILM, which is enriched with heparin sulfate. The heparan sulfate proteoglycan (HSPG) binding region of AAV2.5T is identical to that of AAV5 except for a single substitution of A to T in aa582 of AAV2.5T (aa581 of AAV5) (see FIG. 3A (i-iii)). AAV5 has negligible heparin sulfate binding, where AAV2 has high affinity for heparin sulfate. In order to generate an AAV vector capable of transfecting cells of the retinal following intravitreal injection, a series of swap-variants were created incorporating portions of the AAV2 HSPG binding region into the HSPG binding region of AAV2.5T.

AAV7m8 vectors also have enhanced ability to transduce the retina. In order to further improve the ability of AAV2.5T capsids to transduce the retina, a series of mutants were made that include the 7m8 insert at various positions within the HSPG binding region.

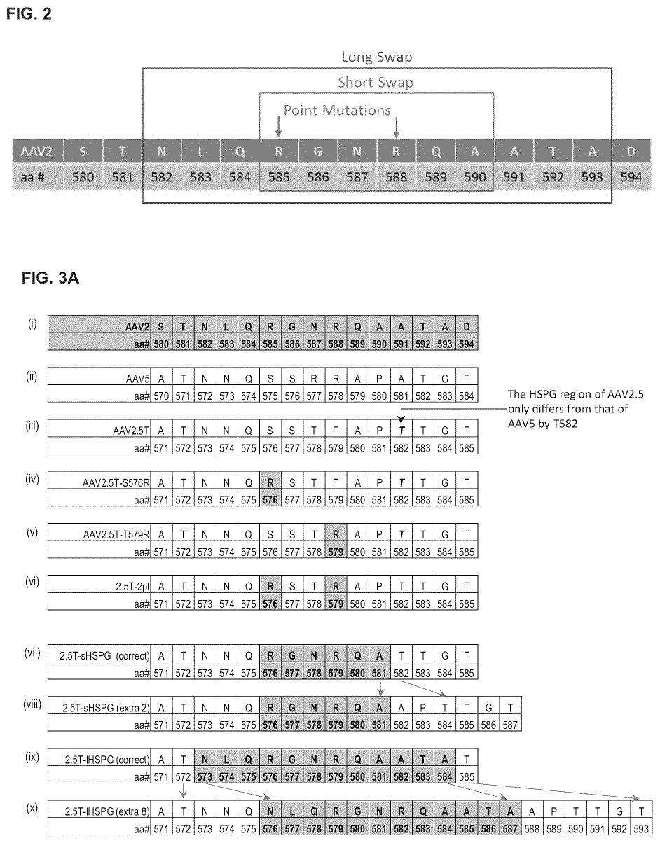

Site-directed mutagenesis and recombinant DNA techniques were used to generate a series of “swap” variants of AAV2.5T, which introduced amino acid residues from the heparan sulfate proteoglycan (HSPG) binding region of AAV2 into AAV2.5T or AAV2.5/7m8 hybrid backbones. The HSPG binding region of AAV2.5T/7m8 variants are shown in FIG. 3B (iv)-(ix). Three types of swap variants were generated: (1) point mutants: (2) short swap variants (sHPSG); and (3) long swap variants (IHSPG). The location of the point mutations and the amino acids transferred in the short swap and long swap mutants are depicted in FIG. 2. FIG. 3A provides a sequence alignment of regions of HSPG swap variants on the 2.5T backbone. FIG. 3B provides alignments of regions of HSPG swap variants on 2.5T/7m8 backbones.

The short AAV2.5T swap variants (sHSPG) were made by replacing amino acid residues corresponding to 576-SSTTAP-581 (SEQ ID NO:26) (sHSPG(correct)) (FIG. 3A(vii)) or 576-SSTT-579 (SEQ ID NO:27) (sHSPG(extra2)) (FIG. 3A(viii)) of 2.5T with amino acid residues 585-RNGRQA-590 (SEQ ID NO:5) of AAV2. The long swap variants (IHSPG) were made by replacing amino acid residues corresponding to 573-584 (IHSPG(correct)) or 576-579 (IHSPG(extra8) of 2.5T with amino acid residues 582-593 of AAV2. Illustrative swap mutants are shown in FIGS. 3A-3B. The double point mutation (S575R+T579R), the sHSPG, and the IHSPG swap variants were made on 2.5T backbone (FIG. 3A), 2.5T/7m8(0) and 2.5T/7m8(−12) backbone (FIG. 3B). Two single mutants, S575R and T579R, were also made to determine if one of the sites was detrimental to the capsid. FIGS. 4A-4C provide the amino acid sequences of the capsid proteins of selected swap variants.

Variants were generated using a triple transfection method of plasmid with ITRs carrying the GFP transgene, a plasmid with the Rep/Cap genes, and a plasmid containing adenovirus-helper functions, followed by ultracentrifugation to separate the empty and full capsids.

The swap variant viruses were characterized by quantitative PCR to establish titer and packaging, and western blot was conducted to ensure the correct ratio of the VP1, VP2 and VP3 capsid proteins.

The majority of the swap variants packaged, resulting in a titer greater than 1×10¹² vector genomes (vg)/ml, and western blots showed the correct ratio of VP1, VP2 and VP3. However, four swap variants did not package and showed much lower titer as compared to others. Table 2 indicates the titer of 13 swap variants, with those exhibiting low titer marked with an asterisk.

TABLE 2
Titer of 2.5T HSPG swap variants

| Vector | Titer (vg/ml) |
|---|---|
| 2.5T-S576R | 4.04E+13 |
| 2.5T-T579R | 5.17E+13 |
| *2.5T-2pt | 1.94E+10, |
| | 4.44E+10 |
| *2.5T-sHSPG | 1.75E+10, |
| | 1.12E+11 |
| *2.5T-lHSPG | 5.11E+11, |
| | 1.45E+11 |
| 2.5T/7m8(+3)- Δ2 | 7E+13 |
| 2.5T/7m8(0)-sHSPG | 6.62E+12 |
| *2.5T/7m8(0)-lHSPG | 4.15E+11, |
| | 2.34E+11 |
| 2.5T/7m8(−12)-Δ2 | 4.6E+12 |
| 2.5T/7m8(−12)-sHSPG2 | 5.01E+12 |
| 2.5T/7m8(−12)-elHSPG | 1.86E+13 |
| 2.5T/7m8(−12)-sHSPG | 9.64E+12 |
| 2.5T/7m8(−12)-lHSPG | 7.76E+12 |

### Example 2

### Heparan Binding Affinity of AAV2.5t Swap Variants

The ability of the swap variants to bind HSPG was determined by performing a heparan binding assay using a pre-packed GE heparan column. Vector was loaded on the column, which was then washed, and finally eluted with increasing concentrations of NaCl (100 mM-1 M). Fractions of Load, Flow-through, Wash, and Elution were collected and analyzed by dot-blot using the B1 antibody.

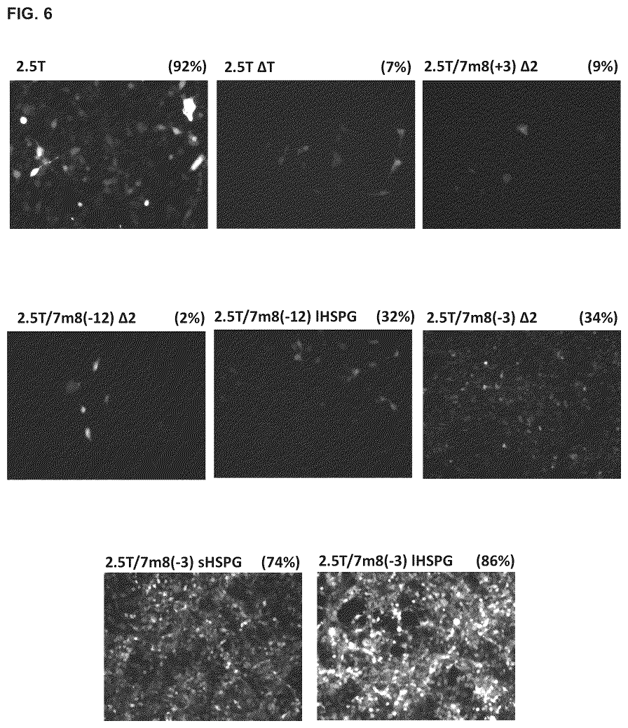

The swap variants demonstrated varying levels of binding affinity to the heparan column. Except for the single mutant, 2.5T-S576R, all of the swap variants gained HSPG binding affinity as compared to 2.5T parent. FIG. 5 shows the binding elution profile as determined by dot-blot.

### Example 3

### Transduction of HEK293 And CHOK1 Cells

In vitro transductions were performed on HEK293 cells or CHOK1 cells at an MOI of 3×10⁵ for five days. Images were captured and flow cytometry was performed at the end of the study.

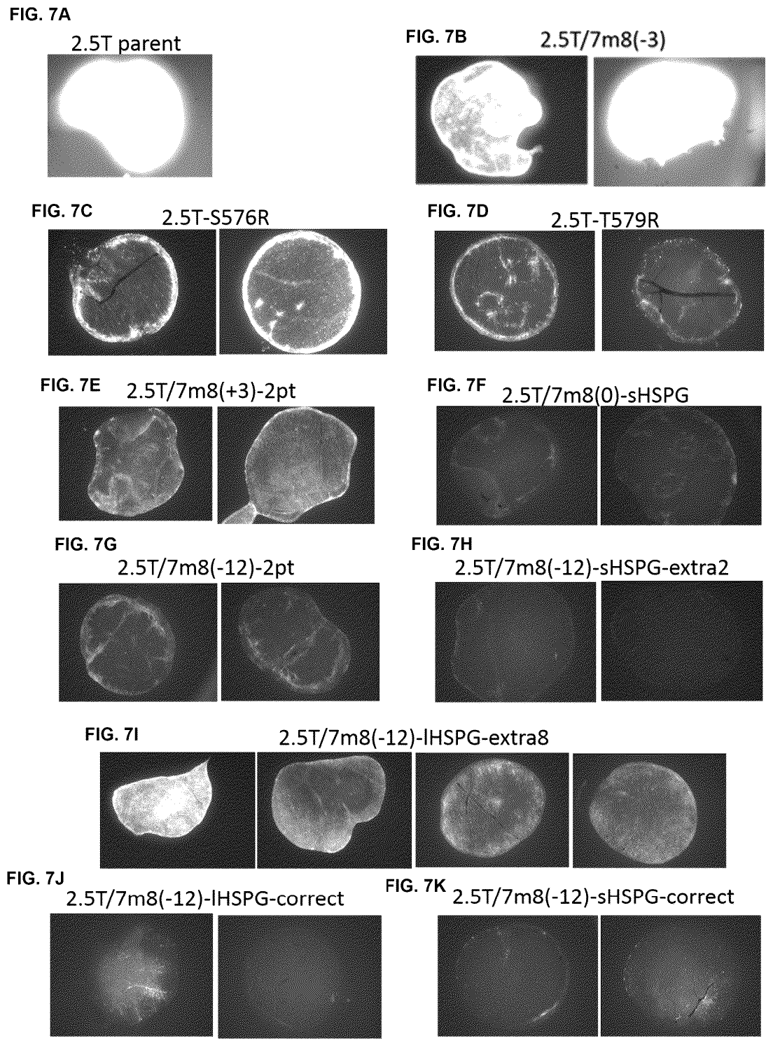

Table 3 provides the in vitro transduction profile of the indicated swap mutants in HEK293 and CHOK1 cells, with the result of a paired AAV2.5T transduction given in parenthesis, and FIG. 6 shows the percent transduction of HEK293 cells achieved by the indicated swap variants. Although the resulting transgene expression was lower as compared to the 2.5T parent, all of the swap variants tested were able to transduce both HEK293 and CHOK1 cells.

TABLE 3
In vitro transduction profile of 2.5T HSPG swap variants

| | Titer | Transduction in | Transduction in |
|---|---|---|---|
| Vector | (vg/ml) | HEK293* | CHOK1 |
| 2.5T-S576R | 4.04E+13 | 65.95% (97.95%) | 93.4% (91.55%) |
| 2.5T-T579R | 5.17E+13 | 7.32% (97.95%) | 48.9% (91.55%) |
| 2.5T/7m8(+3)- | 7E+13 | 9% (80%) | 23.1% (41.2%) |
| Δ2 | | | |
| 2.5T/7m8(0)- | 6.62E+12 | 14% (96%) | 12% (29%) |
| sHSPG | | | |
| 2.5T/7m8(−12)- | 4.6E+12 | 22% (96%) | 4% (29%) |
| Δ2 | | | |
| 2.5T/7m8(−12)- | 5.01E+12 | 21% (96%), | 4% (29%), |
| sHSPG2 | | 5% (97.95%) | 15.85% (91.55%) |
| 2.5T/7m8(−12)- | 1.86E+13 | 69% (96%), | 11% (29%), |
| elHSPG | | 23.1% (80%), | 10.55% (41.2%), |
| | | 36.18% (97.95%) | 46.7% (91.55%) |
| 2.5T/7m8(−12)- | 9.64E+12 | 3.89% (97.95%) | 20.25% (91.55%) |
| sHSPG | | | |
| 2.5T/7m8(−12)- | 7.76E+12 | 7.86% (97.95%) | 24.55% (91.55%) |
| lHSPG | | | |

*results of paired AAV2.5T transduction are given in parenthesis.

### Example 4

### Expression and Tropism in Pig Retinal Explants

Ex vivo pig retinal explants were maintained on trans-wells as described in PCT/US2017/030636, the disclosure of which is incorporated in its entirety, and were transduced with the swap variant viruses at an MOI of 4×10⁴ for two weeks. Live fluorescent images were captured at one and two weeks following transduction.

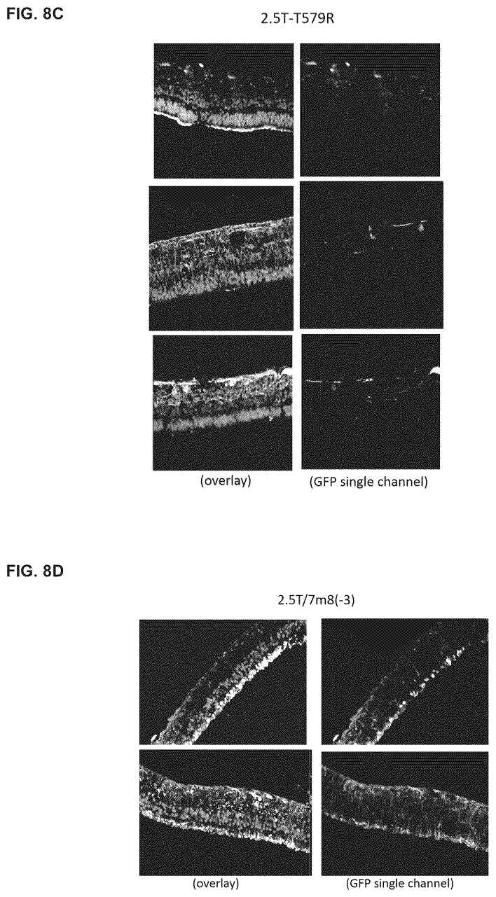

Swap variants displayed variable amounts of gene expression and altered tropism to retinal cells in the pig explants. FIGS. 7A-7K show live imaging of pig retinal explants for the indicated swap mutants. The pig explant samples with higher GFP expression were cryo-sectioned and stained to identify different retinal cell types (FIGS. 8A-8F). Interestingly, all the examined variants showed different tropism from 2.5T parent, even the single point mutants. 2.5T parent restrictedly only infect photoreceptor cells in pig explants (FIG. 8A). Single point mutations 2.5T-S576R and 2.5T-T579R exhibited reduced photoreceptor cell transduction, but were able to transduce RGC cells (FIGS. 8B and 8C). The 2.5T/7m8(+3)-Δ2 vector retained the ability of AAV2.5T to transduce photoreceptor cells, and also exhibited the ability to transduce Müller cells (FIG. 8E). The 2.5T/7m8(−12)-elHSPG vector exhibited variable transduction ability (FIG. 7 panel (I)). Of the four explants transduced by 2.5T/7m8(−12)-elHSPG, only one showed strong transduction of almost all the retinal cells (FIG. 8F, right hand side labeled PEX 1601). However, the other three explants (two from PEX1603 and one from PEX1601) showed much lower transduction levels (a representative image set is shown in FIG. 8F, left hand side).

It was interesting that some swap variants were able to transduce pig retinal explant and actually showed different tropism than the 2.5T parent. For example, 2.5T/7m8(+3)-Δ2 showed a much higher tropism in Müller cells than 2.5T parent, which makes it a potential vector candidate for targeting Müller cells. 2.5T/7m8(−3) had high expression levels in pig explants, and it transduced almost all the retinal cells stained. Therefore, 2.5T/7m8(−3) was chosen as the backbone to build the HSPG double mutant (S576R+T579R), sHSPG swap, and IHSPG swap variants. In addition, HSPG swap variants were also built upon 2.5T/7m8(−12). In particular the 2.5T/7m8(−12) elHSPG showed promising transduction in the pig retinal explants.

### Example 5

### Expression Following Intra Vitreal Administration to Gerbil Retina

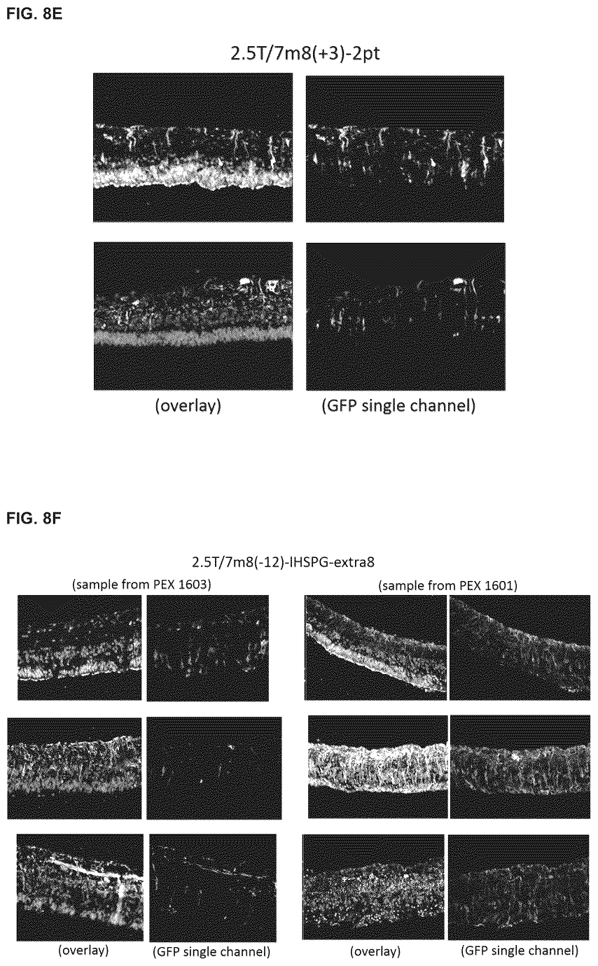

In vivo studies were performed in gerbils, which were intravitreally (IVT) injected with 2E+10 vg/eye of swap variant virus. Fluorescence fundus images were captured at eight weeks following IVT injection (FIG. 9).

Certain swap variants showed significantly improved gene expression in the gerbil retina at eight weeks following IVT administration as compared to parent 2.5T. These results show that certain swap variants may be used to efficiently cross the ILM following IVT administration. As expected, AAV2.5T did not efficiently cross the ILM.

AAV2.5T/7m8(+3)A and AAV2.5T/7m8(−12)elHSPG were able to efficiently cross the ILM and transduce retinal cells following intravitreal injection, and AAV2.5T/7m8(−3)IHSPG exhibited a moderate ability to traverse the ILM.

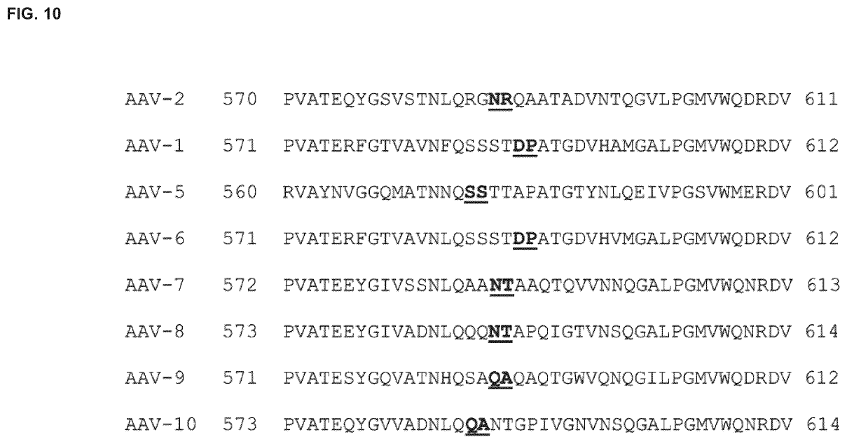

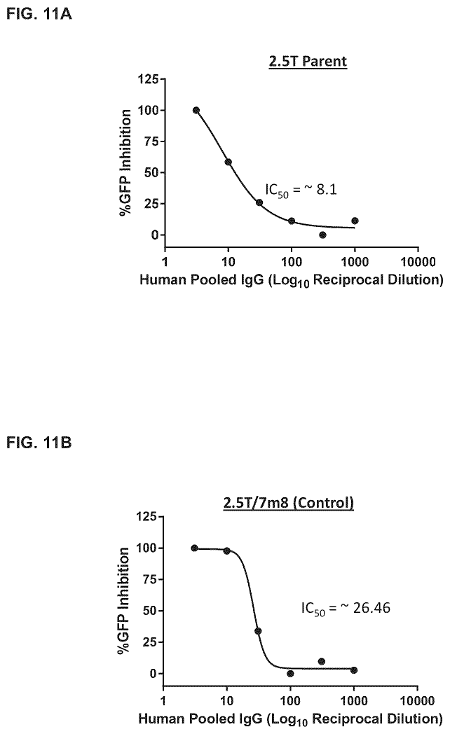

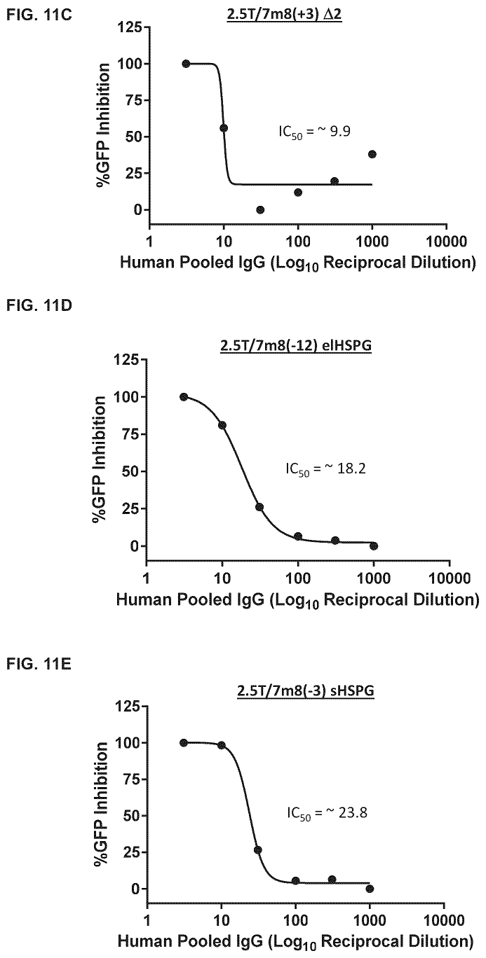

Following fundus imaging, animals were sacrificed, and retinas were removed and cryosectioned. Sections were stained with DAPI, and were probed for GFP, L/M Opsin, parvalbumin, and rhodopsin. FIGS. 12A and 12B provide immunofluorescent images of the retina of a gerbil that received 2.5T/7m8(−12)elHSPG via intravitreal injection. The images show that GFP expression colocalized with parvalbumin and DAPI. FIG. 13 provides and immunofluorescent image of the retina of a gerbil that received 2.5T/7m8(−3)IHSPG. The image shows that GFP expression colocalized with parvalbumin and DAPI. FIGS. 14A and 14B provide immunofluorescent images of the retina of a gerbil that received 2.5T/7m8(+3) Δ2. The images show that GFP expression colocalized with parvalbumin and DAPI.

### Example 6

### Determination of Neutralizing Antibody Profile

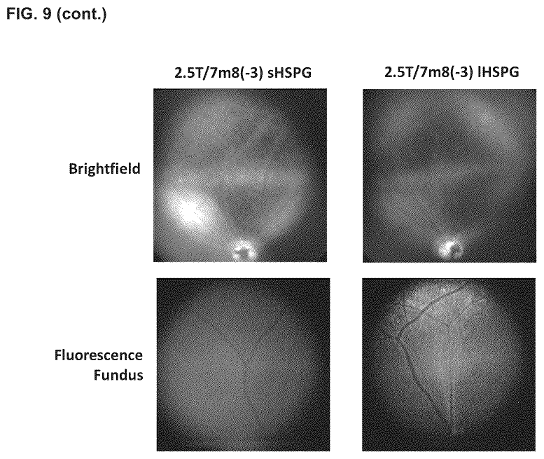

Neutralizing antibody (nAB) profiles were determined for swap variant virus by in vitro assay. Briefly, seven 1:3.1-fold serial dilutions of pooled human IgG antibodies (Gammaguard IVIG) were prepared (1:1, 1:3.1, 1:10, 1:31, 1:100, 1:310, and 1:1000). IgG serial dilutions were combined with swap variant virus carrying GFP, and used to transduce 293T cells at MOI of 1E+5 vg/cell. Transductions were carried out in quadruplicate. At 72 hours post-transduction, GFP expression was measured. Results are reported in FIGS. 11A-11E. Swap variants were shown to have a similar nAb profile to the AAV2.5T parent vector, which is improved over AAV2 (compare FIG. 1B top panel with FIGS. 11A-11E).

### Example 7

### Expression Following Intra Vitreal Administration to Non-Human Primate Retina

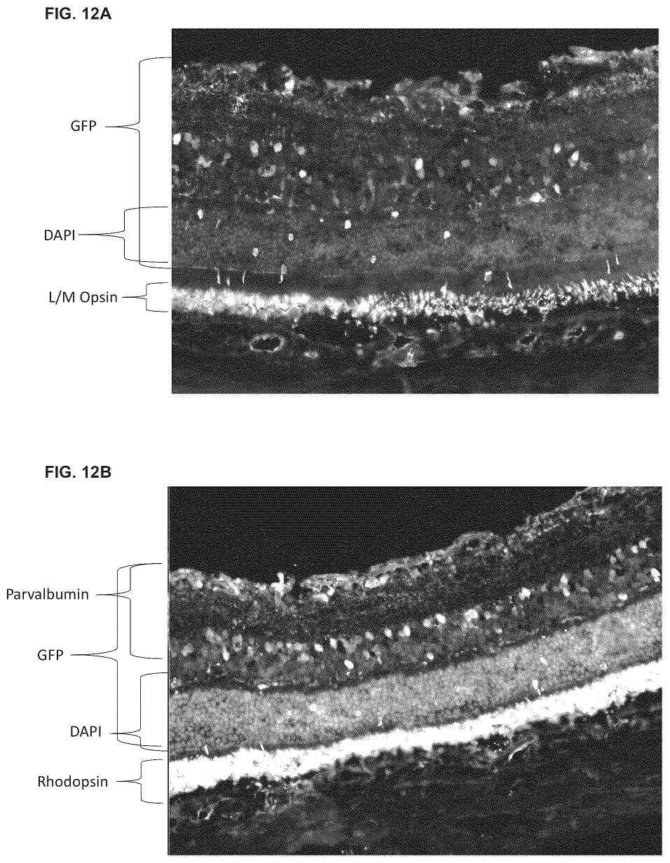

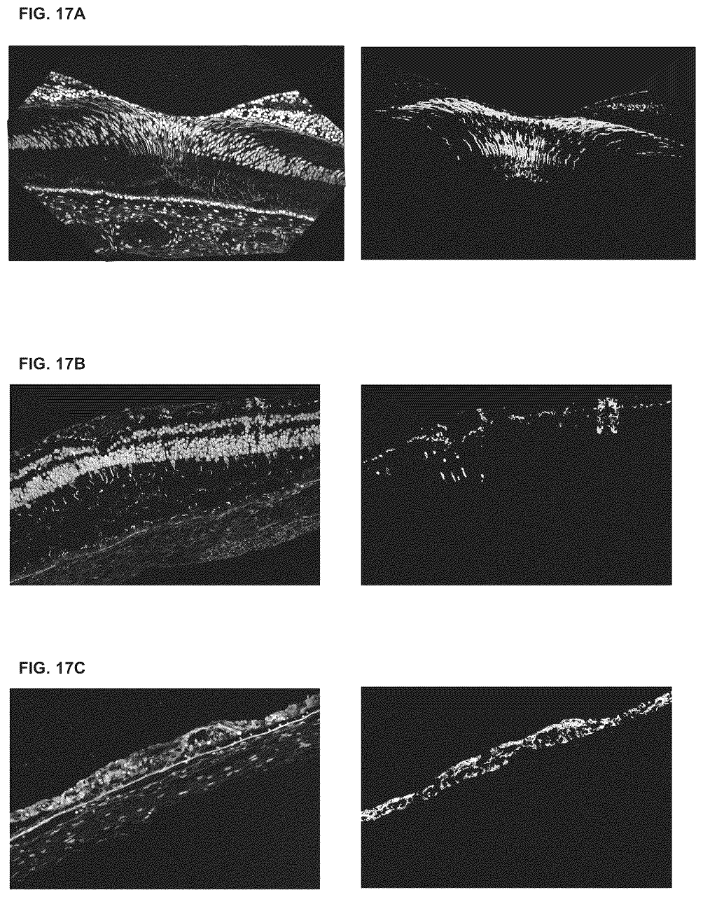

In vivo studies were performed in green monkeys (Chlorocebus sabaeus), which were intravitreally injected with 2.5E+12 vg/eye of swap variant viruses AAV2.5T/7m8(−12)elHSPG and AAV2.5T/7m8(+3)Δ2. OTC autofluorescence images were captured using a SPECTRALIS OCT (Heidelberg Engineering) at twelve weeks post injection and are shown in FIG. 15 (AAV2.5T/7m8(−12)elHSPG), and FIG. 19 (AAV2.5T/7m8(+3)Δ2). Unexpectedly, AAV2.5T/7m8(−12)elHSPG exhibited expression in the macula as well as inside the blood arcades. The blood arcade region of the retina has the thickest ILM, which is thought to block transduction of retinal cells by most AAV when injected intravitreally.

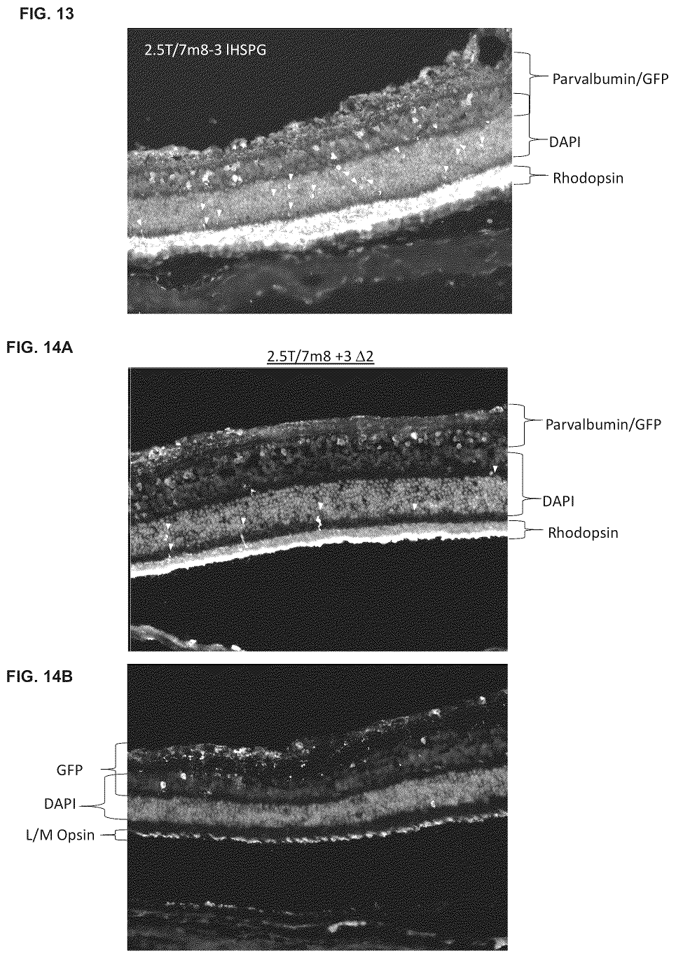

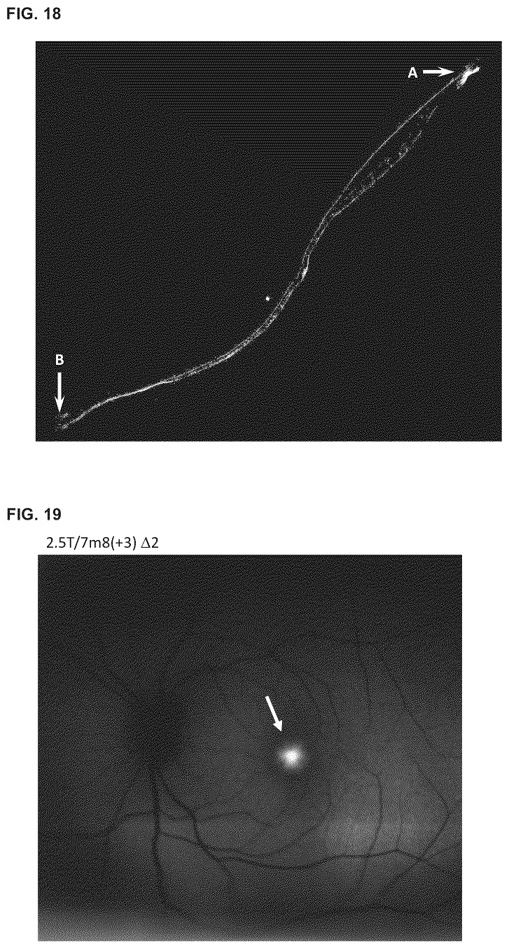

Following OTC autofluorescence, animals were sacrificed, and retinas were removed. Retina flat mounts were made from animals injected with swap variant viruses. Live imaging of flat mounted retinas is depicted in FIG. 16 (2.5T/7m8(−12)elHSPG) and FIG. 20 (2.5T/7m8(+3)Δ2). 2.5T/7m8(−12)elHSPG exhibited expression throughout the retina, whereas expression from 2.5T/7m8(+3)Δ2 was concentrated in the macula.

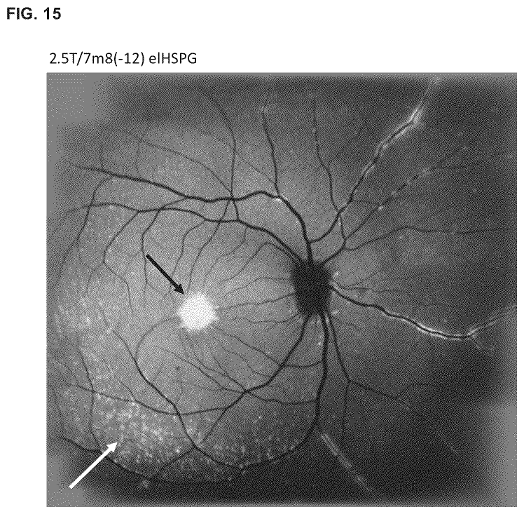

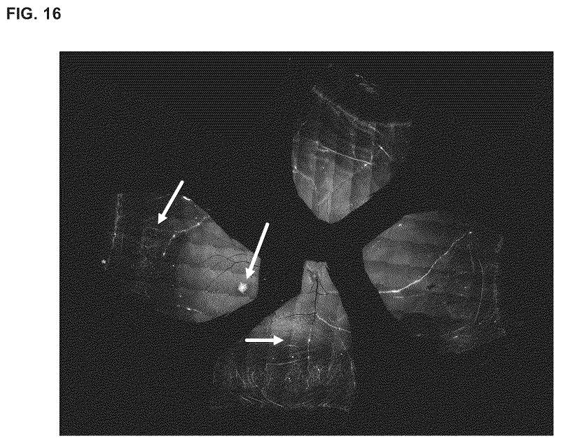

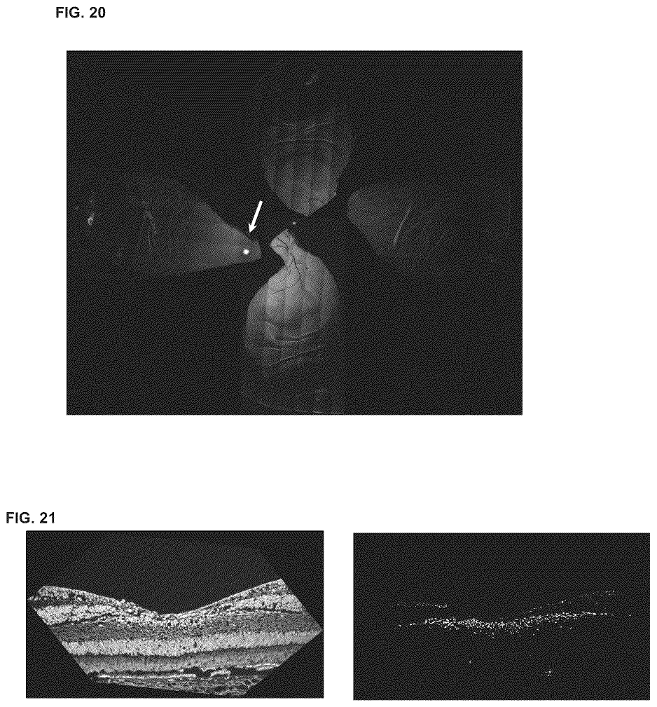

Next, retinas were cryo-sectioned to obtain transverse sections at the points indicated by the arrows on the flat mount images. Immunofluorescent imaging confirmed that 2.5T/7m8(−12)elHSPG was successfully able to penetrate the ILM and transduce cells from the fovea to the ora serrata, as evidenced by the GFP expression shown in FIG. 18, as well as transverse sections taken at the fovea (FIG. 17A) mid-periphery (FIG. 17B) and periphery (FIG. 17C). which show robust GFP expression in RGC. cones. and Müller cells. Immunofluorescent imaging confirmed that 2.5T/7m8(+3)Δ2 primarily transduces Müller cells in the fovea of non-human primates (FIGS. 20 and 21).

1. A non-naturally-occurring modified AAV capsid protein, comprising one or more amino acid modifications, wherein the modification confers heparan sulfate binding to the capsid protein.

2. The modified AAV capsid protein of claim 1, wherein the AAV is an AAV2.5T.

3. The modified AAV capsid protein of claim 1, wherein the AAV is an AAV2.5T/7m8.

4. The modified AAV capsid protein of claim 3, wherein the AAV2.5T/7m8 is an AAV2.5T/7m8(+3), an AAV2.5T/7m8(0), an AAV2.5T/7m8(−12), or an AAV2.5T/7m8(−3).

5. The modified AAV2.5T capsid protein of claim 1 or claim 2, wherein at least one of the one or more amino acid modifications comprises:
(a) a S576R point mutation;
(b) a T579R point mutation;
(c) a substitution of amino acid residues 576-579 or 576-581 with the following amino acid residues: RGNRQA (SEQ ID NO:5);
(d) a substitution of amino acid residues 576-581 with the following amino acid residues: RGNRQAAP (SEQ ID NO:6);
(e) a substitution of amino acid residues 573-579, 573-581 or 573-584 with the following amino acid residues: NLQRGNRQAATA (SEQ ID NO:7); or
(f) a substitution of amino acid residues 573-581 or 573-584 with the following amino acid residues: NLQRGNRQAATAAP (SEQ ID NO:8).

6. The modified AAV2.5T/7m8 capsid protein of any of claim 1, 3 or 4, wherein at least one of the one or more amino acid modifications comprises:
(a) a point mutation corresponding to a S576R point mutation in AAV2.5T;
(b) a point mutation corresponding to a T579R point mutation in AAV2.5T;
(c) a substitution corresponding to a substitution of amino acid residues 576-579 or 576-581 in AAV2.5T with the following amino acid residues: RGNRQA (SEQ ID NO:5);
(d) a substitution corresponding to a substitution of amino acid residues 576-581 in AAV2.5T with the following amino acid residues: RGNRQAAP (SEQ ID NO:6);
(e) a substitution corresponding to a substitution of amino acid residues 573-579, 573-581, 573-583, 573-584, 576-579, 576-581, 576-583 or 576-584 in AAV2.5T with the following amino acid residues: NLQRGNRQAATA (SEQ ID NO:7); or
(f) a substitution corresponding to a substitution of amino acid residues 576-583, 576-584, 573-583 or 573-584 in AAV2.5T with the following amino acid residues: NLQRGNRQAATAAP (SEQ ID NO:8).

7. The modified AAV2.5T/7m8 capsid protein of claim 6, comprising a capsid sequence set forth in any of SEQ ID NOs:1-3.

8. The modified AAV2.5T or AAV2.5T/7m8 capsid protein of claim 1, comprising a sequence shown in FIGS. 3A-3C.

9. A polynucleotide comprising a nucleic acid sequence encoding the modified AAV2.5T or AAV2.5T/7m8 capsid protein of any of claims 1-8.

10. An expression vector comprising the polynucleotide of claim 9, wherein the nucleic acid sequence encoding the modified AAV2.5 or AAV2.5T is operably linked to a promoter sequence.

11. A cell comprising the expression vector of claim 10.

12. The cell of claim 11, further comprising a polynucleotide that encodes a therapeutic protein.

13. The cell of claim 10 or claim 12, further comprising a polynucleotide that encodes a rep protein.

14. A recombinant virus or viral vector comprising the modified capsid protein of any one of claims 1-8.

15. The recombinant virus or viral vector of claim 14, wherein the recombinant virus or viral vector is an adeno-associated virus (AAV), optionally AAV2.

16. The recombinant virus or viral vector of claim 14 or claim 15, wherein the recombinant virus is eluted from a heparan column at a salt concentration of about 0.2 M to about 0.4 M.

17. The recombinant virus or viral vector of any of claims 14-16, wherein the recombinant virus or viral vector is capable of binding to and crossing the inner limiting membrane (ILM) when intravitreally injected into a mammal.

18. The recombinant virus of any one of claims 14-17, wherein the recombinant virus or viral vector comprises a polynucleotide sequence that encodes a therapeutic protein.

19. The recombinant virus or viral vector of claim 18, wherein the therapeutic protein is an anti-vascular endothelial growth factor (anti-VEGF) agent.

20. The recombinant virus or viral vector of any one of claims 14-18, wherein the recombinant virus or viral vector has an altered cellular tropism as compared to AAV2.5T or AAV2.5T/7m8.

21. The recombinant virus or viral vector of claim 20, wherein the recombinant virus or viral vector is a AAV2.5T comprising a modified capsid protein comprising: (a) a S576R point mutation: or (b) a T579R point mutation, wherein the recombinant virus or viral vector has a greater tropism for RGC than AAV2.5T.

22. The recombinant virus of claim 20, wherein the recombinant virus or viral vector is a AAV2.5T/7m8 comprising a modified capsid protein comprising: (a) a S576R point mutation: and (b) a T579R point mutation, wherein the recombinant virus or viral vector has a greater tropism for Müller cells than AAV2.5T/7m8.

23. The recombinant virus or viral vector of claim 20, wherein the recombinant virus or viral vector is a AAV2.5T/7m8 comprising a modified capsid protein comprising a substitution of amino acid residues corresponding to 573-584 in AAV2.5T/7m8 with the following amino acid residues: NLQRGNRQAATA (SEQ ID NO:7), wherein the recombinant virus or viral vector has a greater tropism for retinal cells than AAV2.5T/7m8.

24. A pharmaceutical composition comprising the recombinant virus or viral vector of any one of claims 14-23.

25. A method of providing a protein to a retina of a subject, comprising administering to the subject by intravitreal injection the recombinant virus or viral vector of any one of claims 14-23 or the pharmaceutical composition of claim 24, wherein the recombinant virus or viral vector comprises a polynucleotide sequence that encodes the protein.

26. A method of providing a therapeutic protein to a retina of a subject in need thereof, comprising administering to the subject by intravitreal injection a pharmaceutical composition comprising the recombinant virus or viral vector of any one of claims 18-23.

27. The method of claim 26, wherein the subject has been diagnosed with or is suspected of having one or more conditions selected from the group consisting of: age-related macular degeneration (AMD), wet-AMD, dry-AMD, retinal neovascularization, choroidal neovascularization, diabetic retinopathy, proliferative diabetic retinopathy, retinal vein occlusion, central retinal vein occlusion, branched retinal vein occlusion, diabetic macular edema, diabetic retinal ischemia, ischemic retinopathy, and diabetic retinal edema.

28. A method of altering the tropism of an AAV2.5T or AAV2.5T/7m8 virus or viral vector, comprising introducing one or more amino acid modifications that confers heparan sulfate binding to a capsid protein of the virus or viral vector.

29. The method of claim 28, wherein the amino acid modification introduces one or more amino acids of AAV2 into the capsid protein.

30. The method of claim 28 or claim 29, wherein at least one of the one or more amino acid modifications comprises:
(a) a point mutation corresponding to a S576R point mutation in AAV2.5T;
(b) a point mutation corresponding to a T579R point mutation in AAV2.5T;
(c) a substitution corresponding to a substitution of amino acid residues 576-581 in AAV2.5T with amino acid residues: RGNRQA (SEQ ID NO:5) or RGNRQAAP (SEQ ID NO:6); or
(d) a substitution corresponding to a substitution of amino acid residues 573-584 in AAV2.5T with amino acid residues: NLQRGNRQAATA (SEQ ID NO:7) or NLQRGNRQAATAAP (SEQ ID NO:8).

31. The method of claim 29, wherein the amino acid modification comprises: (a) a S576R point mutation; or (b) a T579R point mutation, wherein the virus or viral vector has a greater tropism for RGC than AAV2.5T.

32. The method of claim 29, wherein the amino acid modification comprises: (a) a S576R point mutation; and (b) a T579R point mutation, wherein the virus or viral vector has a greater tropism for Müller cells than AAV2.5T/7m8.

33. The method of claim 29, wherein the amino acid modification comprises a substitution of amino acid residues corresponding to 573-584 in AAV2.5T/7m8 with the following amino acid residues: NLQRGNRQAATA (SEQ ID NO:7), wherein the recombinant virus or viral vector has a greater tropism for retinal cells than AAV2.5T/7m8.

34. A method for selectively delivering a polypeptide to RGC of a subject, comprising administering to the subject an AAV2.5T virus or viral vector comprising a polynucleotide that encodes the polypeptide, wherein the virus or viral vector comprises a capsid protein comprising a S576R point mutation or a T579R point mutation.

35. A method for selectively delivering a polypeptide to Müller cells of a subject, comprising administering to the subject an AAV2.5T/7m8 virus or viral vector comprising a polynucleotide that encodes the polypeptide, where the virus comprises a capsid protein comprising: (a) a S576R point mutation; and (b) a T579R point mutation, optionally AAV2.5T/7m8(+3)Δ2.

36. A method for selectively delivering a polypeptide to retinal cells of a subject, comprising administering to the subject an AAV2.5T/7m8 virus or viral vector comprising a polynucleotide that encodes the polypeptide, where the virus comprises a capsid protein comprising a substitution of amino acid residues corresponding to 573-583 in AAV2.5T/7m8 with amino acid residues: NLQRGNRQAATA (SEQ ID NO:7) or NLQRGNRQAATAAP (SEQ ID NO:8).
